(19) United States
(12) Patent Application Publication
(10) Pub. No.: US 2026/0232636 A1
(43) Pub. Date: Aug. 13, 2026

(54) INHIBITORS OF LRRK2

(71) Applicant: Vanderbilt University, Nashville, TN (US)

(72) Inventors: Craig W. Lindsley, Brentwood, TN (US); Changho Han, Nashville, TN (US); Upendra Rathnayake, Nashville, TN (US); Joseph D. Bungard, Nashville, TN (US); Hyekyung Kelly Plumley, Nashville, TN (US)

(21) Appl. No.: 19/153,251

(22) PCT Filed: Mar. 1, 2024
(86) PCT No.: PCT/US2024/018036

§ 371 Date: Aug. 1, 2025

Related U.S. Application Data
(60) Provisional application No. 63/487,792, filed on Mar. 1, 2023.

(51) Int. Cl. A61K 31/416; A61K 31/433; A61K 31/454; C07D 401/14; C07D 403/14; C07D 405/14; C07D 409/14; C07D 417/14

(52) U.S. Cl. CPC A61K 31/416; A61K 31/433; A61K 31/454; C07D 401/14; C07D 403/14; C07D 405/14; C07D 409/14; C07D 417/14

(57) ABSTRACT

Indazole- and azaindazole-substituted octahydrocyclopenta[c]pyrrole and hexahydrocyclopenta[c]pyrrole compounds are inhibitors of LRRK2. The compounds and their pharmaceutical compositions may be used in the treatment of CNS disorders such as familial/genetic and/or sporadic Parkinson's disease, a tauopathy, or Alzheimer's disease.

### RELATED APPLICATIONS

This application claims priority to U.S. Provisional Application No. 63/487,792, filed Mar. 1, 2023, which is hereby incorporated by reference in its entirety.

### TECHNICAL FIELD

The present invention is directed to compounds which are selective inhibitors of G2019S mutant LRRK2 over Wild-Type LRRK2 and are useful in the treatment of CNS disorders, such as familial/genetic and/or sporadic Parkinson's disease, as well as certain cancers, Crohn's disease, Alzheimer's disease and leprosy.

### BACKGROUND

Parkinson's disease (“PD”) is a chronic, progressive movement disorder (1-2% of population over age 65) characterized by tremor, bradykinesia, impaired speech, postural abnormalities and poor quality of life. There are no disease modifying treatments at present, only palliative treatment of the dysregulated movement with dopamine replacement therapies; however, these are plagued with ‘on’/‘off’ cycles and adverse events related to hyperdopaminergic states. Thus, new, non-dopaminergic approaches to treat PD represent an unmet medical need.

Leucine-rich repeat kinase 2 (LRRK2) is a critical influencing factor for PD, and has recently been implicated in PD by genome-wide association studies. Furthermore, dysfunction of LRRK2 may influence the accumulation of α-synuclein and signaling pathways by the kinase activation of LRRK2, and LRRK2 accumulates in Lewy bodies (Curr Neuropharmacol. 2018 November; 16(9): 1348-1357; (Eur. J Neurosci. 2006, 23(3):659)). Many PD cases are sporadic while up to 10% of cases are inherited with mutations identified in several genes in families, such as LRRK2 and the G2019S mutation. Patients with point mutations, such as the G2019S LRRK2, present PD pathology indistinguishable from idiopathic patients. Despite the identification of more than 20 LRRK2 mutations associated with autosomal-dominantly inherited parkinsonism, the G2019S mutation, found within the kinase domain of LRRK2, accounts for >85% of LRRK2-associated PD patients. Moreover, G2019S is an activating mutation, and thus, inhibitors of G2019S LRRK2 are a therapeutic approach for the treatment of PD patients harboring the G2019S mutation. In addition, LRRK2 is associated with many other diseases such as tauopathies, certain cancers, Crohn's disease, Alzheimer's disease and leprosy (BMB Rep 2015 May; 48(5):243-8).

LRRK2 mutations have been associated with Alzheimer's-like pathology (Zimprach et al., Neuron. 2004 Nov. 18; 44(4):601-7). The LRRK2 R1628P variant has been associated with an increased risk of developing Alzheimer's disease (AD) (Zhao et al., Neurobiol Aging. 2011 November; 32(11): 1990-3). Mutations in LRRK2 have been identified that are clinically associated with the transition from mild cognitive impairment to AD (see WO2007149798). These data suggest that LRRK2 inhibitors may be useful in the treatment of AD and other dementias and related neurodegenerative disorders.

LRRK2 has been reported to phosphorylate tubulin-associated tau and the phosphorylation is enhanced by the kinase activating LRRK2 mutation G2019S (Kawakami et al., PLoS One. 2012; 7(1):e30834; Bailey et al., Acta Neuropathol. 2013 December; 126(6):809-27). Overexpression of LRRK2 in a tau transgenic mouse model resulted in the aggregation of insoluble tau and its phosphorylation at multiple epitopes (Bailey et al., 2013). Hyperphosphorylation of tau has been observed in LRRK2 R1441G overexpressing transgenic mice (Li et al., NatNeurosci. 2009 July; 12(7):826-8). Inhibition of LRRK2 kinase activity may thus be useful in treating tauopathy disorders characterized by hyperphosphorylation of tau, e.g., argyrophilic grain disease, Picks disease, corticobasal degeneration, progressive supranuclear palsy, inherited frontotemporal dementia and Parkinson's linked to chromosome 17 (Goedert and Jakes, Biochim Biophys Acta. 1739, (2005) 240-250).

Evidence suggests a role for LRRK2 in immune cell function in the brain, with LRRK2 inhibitors demonstrated to attenuate microglial inflammatory responses (Moehle et al., J Neurosci. 2012 Feb. 1; 32(5): 1602-11). Significantly elevated levels of LRRK2 mRNA have been observed in muscle biopsy samples taken from patients with ALS (Shtilbans et al., Amyotroph Lateral Scler. 2011 July; 12(4):250-6). As neuroinflammation is a hallmark of some neurodegenerative diseases such PD, AD, MS, HIV-induced dementia, ALS, ischemic stroke, traumatic brain injury and spinal cord injury, LRRK2 kinases inhibitors may have utility in the treatment of neuroinflammation in these disorders.

LRRK2 is expressed in cells of the immune system and studies suggest that LRRK2 may play a role in the regulation of the immune system and modulation of inflammatory responses. LRRK2 kinase inhibitors may thus be of utility in a number of diseases of the immune system such as lymphomas, leukemias, multiple sclerosis rheumatoid arthritis, systemic lupus erythematosus autoimmune hemolytic anemia, pure red cell aplasia, idiopathic thrombocytopenic pupura (ITP), Evans Syndrome, vasculitis, bullous skin disorder, type I diabetes mellitus, Sjogren's syndrome, Delvic's disease, inflammatory myopathies (Engel at al., Pharmacol Rev. 2011 March; 63(1): 127-56; Homam et al., Clin Neuromuscular disease, 2010) and ankylosing spondylitis (Danoy et al., PLoS Genet. 2010 Dec. 2; 6(12)).

Increased incidence of certain types of non-skin cancers such as renal, breast, lung, prostate, and acute myelogenous leukemia (AML) have been reported in patients with the LRRK2 G2019S mutation (Agalliu et al., JAMA Neurol. 2015 January; 72(1); Saunders-Pullman et al., Mov Disord. 2010 Nov. 15; 25(15):2536-41). LRRK2 amplification and overexpression has been reported in papillary renal and thyroid carcinomas. LRRK2 also significantly correlated with increased risk of Low-Grade Glioma (Yan et al., Genomics 2022, 114: 316-327). Inhibiting LRRK2 kinase activity may therefore be useful in the treatment of cancer (Looyenga et al., Proc Natl Acad Sci USA 2011 Jan. 25; 108(4):1439-44).

Genome-wide association studies also highlight LRRK2 in the modification of susceptibility to the chronic autoimmune Crohn's disease and leprosy (Zhang et al., The New England Journal of Medicine, Vol 361, 2009, pp. 2609-2618; Umeno et al., Inflammatory Bowel Disease Vol 17, 2011, pp. 2407-2415).

The challenge in the field is to develop inhibitors selective for the G2019S mutant LRRK2 over wild-type LRRK2. Numerous preclinical and clinical studies have shown that inhibition of WT LRRK2 in the periphery induces fibrosis in lung and other tissues, thus lowering the therapeutic index. Non-selective LRRK2 inhibitors induce reversible changes in non-human primate lungs without measurable pulmonary deficit (Sci Transl Med. 2020 Apr. 22; 12(540)), indicating that for use as chronic therapy in PD patients, as well as other chronic indications, high selectivity for the G2019S mutant LRRK2 is required. To date, there have been few reports of inhibitors with high selectivity for the G2019S mutant LRRK2, and thus a critical need remains for developing selective G2019S LRRK2 inhibitors.

### SUMMARY

One aspect of the invention provides compounds of formula (I),

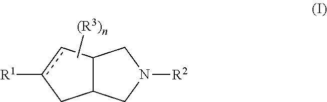

or a pharmaceutically acceptable salt thereof, wherein:
“ [symbol] ” represents a single bond or a double bond;
R¹ is

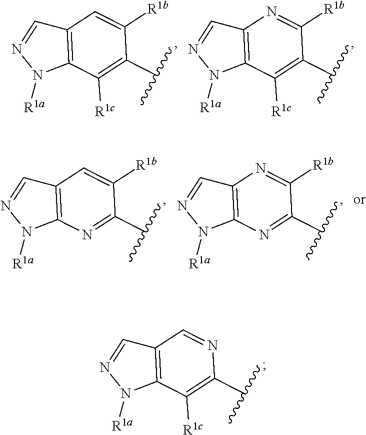

R^{1a} is G¹, -L¹-G¹, —C₂₋₄alkylene-OR^{A}, —C₂₋₄alkylene-NR^{A}R^{B}, C₁₋₆alkyl, C₁₋₆fluoroalkyl, or H;
R^{A} is G¹, -L¹-G¹, C₁₋₆alkyl, C₁₋₆fluoroalkyl, C₃₋₆cycloalkyl, or —C₁₋₃alkylene-C₃₋₆cycloalkyl;
R^{B} is H, C₁₋₆alkyl, C₁₋₆fluoroalkyl, C₃₋₆cycloalkyl, or —C₁₋₃alkylene-C₃₋₆cycloalkyl;
R^{1b} is halogen, C₁₋₄alkyl, C₁₋₂fluoroalkyl, CN, C₃₋₄cycloalkyl, —OC₁₋₃alkyl, —OC₁₋₂fluoroalkyl, or H;
R^{1c} is H, halogen, C₁₋₄alkyl, C₁₋₂fluoroalkyl, CN, phenyl, C₃₋₄cycloalkyl, —OR^{1d}, or —N(R^{1d})₂;
R^{1d}, at each occurrence, is independently H, C₁₋₄alkyl, C₁₋₂fluoroalkyl, C₃₋₄cycloalkyl, or —C₁₋₃alkylene-C₃₋₄cycloalkyl, wherein alternatively two R^{1d}, together with a nitrogen to which the two R^{1d} attach form a 4- to 6-membered heterocyclic ring optionally substituted with 1-4 substituents independently selected from the group consisting of halogen and C₁₋₄alkyl;
L¹ is C₁₋₅alkylene or C₂₋₅alkenylene;
G¹ is a 5- to 6-membered aromatic or partially unsaturated heterocyclic ring containing a first nitrogen and optionally 1-2 additional heteroatoms that are independently nitrogen, oxygen, or sulfur, the heterocyclic ring being attached at an unsaturated carbon in the heterocyclic ring or G¹ is phenyl, G¹ being optionally substituted with a first substituent selected from the group consisting of halogen, C₁₋₄alkyl, cyano, C₁₋₂fluoroalkyl, oxo, —OR¹⁰, —N(R¹⁰)₂, —C₁₋₃alkylene-OR¹⁰, C₃₋₅cycloalkyl, and —C₁₋₃alkylene-C₃₋₅cycloalkyl, and optionally further substituted with 1-2 substituents independently selected from the group consisting of halogen, C₁₋₄alkyl, cyano, and C₁₋₂fluoroalkyl;
R¹⁰, at each occurrence, is independently H, C₁₋₄alkyl, C₁₋₂fluoroalkyl, C₃₋₄cycloalkyl, or —C₁-3alkylene-C₃₋₄cycloalkyl, wherein alternatively two R¹⁰, together with a nitrogen to which the two R¹⁰ attach form a 4- to 6-membered heterocyclic ring optionally substituted with 1-4 substituents independently selected from the group consisting of halogen and C₁₋₄alkyl;
R² is G², -L²-G², -L³-C₁₋₆alkyl, —C₂₋₆alkylene-R^{2a}, C₁₋₆alkyl, C₁₋₆fluoroalkyl, or H;
L² is C₁₋₃alkylene or L³;
L³ is C(O), SO₂, S(O)(NH), C(O)NH, or C(O)O;
G² is a 4- to 12-membered heterocyclyl, a C₃₋₁₂carbocyclyl, a 5- to 12-membered heteroaryl, or a 6- to 12-membered aryl, wherein G² is optionally substituted with a first substituent selected from the group consisting of halogen, cyano, C₁₋₄alkyl, C₁₋₂fluoroalkyl, G^{2a}, oxo, —OR¹³, —N(R¹³)₂, —C₁₋₃alkylene-OR¹³, —C₁₋₃alkylene-N(R¹³)₂, —C(O)N(R¹³)₂, —C(O)OR¹³, —SO₂R¹, and S(O)(NH)R¹³, optionally further substituted with oxo, and optionally further substituted with 1-3 substituents independently selected from the group consisting of halogen, cyano, C₁₋₄alkyl, and C₁₋₂fluoroalkyl;
G^{2a} is a C₃₋₄cycloalkyl;
R³, at each occurrence, is independently H, C₁₋₄alkyl, C₁₋₂fluoroalkyl, C₃₋₄cycloalkyl, or —C₁-3alkylene-C₃₋₄cycloalkyl, wherein alternatively two R¹³, together with a nitrogen to which the two R¹³ attach form a 4- to 6-membered heterocyclic ring optionally substituted with 1-4 substituents independently selected from the group consisting of halogen and C₁₋₄alkyl;
R^{2a} is —OR¹⁴, —N(R¹⁴)₂, —SO₂R¹⁴, S(O)(NH)R¹⁴, —NR¹⁴C(O)N(R¹⁴)₂, or —NR¹⁴C(O)OR¹⁴;
R¹⁴, at each occurrence, is independently H, C₁₋₄alkyl, C₁₋₂fluoroalkyl, C₃₋₄cycloalkyl, or —C₁₋₃alkylene-C₃₋₄cycloalkyl, wherein alternatively two R¹⁴, together with a nitrogen to which the two R¹⁴ attach form a 4- to 6-membered heterocyclic ring optionally substituted with 1-4 substituents independently selected from the group consisting of halogen and C₁₋₄alkyl;
R³, at each occurrence, is independently fluoro or C₁₋₄alkyl; and
n is 0, 1, or 2.

In another aspect, the invention provides a pharmaceutical composition comprising a compound of formula (I), or a pharmaceutically acceptable salt thereof, and a pharmaceutically acceptable carrier.

In another aspect, the invention provides a method of treating a disease or disorder in a subject, wherein the subject would benefit from inhibition of LRRK2, comprising administering to the subject a therapeutically effective amount of a compound of formula (I), or a pharmaceutically acceptable salt or composition thereof.

In another aspect, the invention provides a method for inhibiting LRRK2 in a subject, comprising administering to the subject a therapeutically effective amount of a compound of formula (I), or a pharmaceutically acceptable salt or composition thereof.

In another aspect, the invention provides a method for the treatment of a disease or disorder comprising administering to a subject in need thereof a therapeutically effective amount of a compound of formula (I), or a pharmaceutically acceptable salt or composition thereof, wherein the disease or disorder is a CNS disorder, an immunological disorder, a cancer, or leprosy.

In another aspect, the invention provides a method for the treatment of a CNS disease or disorder such as familial/genetic and/or sporadic Parkinson's disease, a tauopathy, or Alzheimer's disease comprising administering to a subject in need thereof, a therapeutically effective amount of a compound of formula (I), or a pharmaceutically acceptable salt or composition thereof.

In another aspect, the invention provides a compound of formula (I), or a pharmaceutically acceptable salt or composition thereof, for use in the treatment of a disease or disorder selected from the group consisting of a CNS disorder, an immunological disorder, a cancer, and leprosy.

In another aspect, the invention provides a compound of formula (I), or a pharmaceutically acceptable salt or composition thereof, for use in the treatment of a CNS disease or disorder such as familial/genetic and/or sporadic Parkinson's disease, a tauopathy, or Alzheimer's disease.

In another aspect, the invention provides a compound of formula (I), or a pharmaceutically acceptable salt or composition thereof, for use in inhibiting LRRK2 in a subject.

In another aspect, the invention provides the use of a compound of formula (I), or a pharmaceutically acceptable salt or composition thereof, in the manufacture of a medicament for the treatment of a disease or disorder selected from the group consisting of a CNS disorder, an immunological disorder, a cancer, and leprosy.

In another aspect, the invention provides the use of a compound of formula (I), or a pharmaceutically acceptable salt or composition thereof, in the manufacture of a medicament for the treatment of a CNS disease or disorder such as familial/genetic and/or sporadic Parkinson's disease, a tauopathy, or Alzheimer's disease.

In another aspect, the invention provides the use of a compound of formula (I), or a pharmaceutically acceptable salt or composition thereof, in the manufacture of a medicament for inhibiting LRRK2 in a subject.

In another aspect, the invention provides a kit comprising a compound of formula (I), or a pharmaceutically acceptable salt or composition thereof, and instructions for use.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched chain saturated hydrocarbon. Representative examples of alkylene include, but are not limited to, —CH₂—, —CD₂-, —CH₂CH₂—, —C(CH₃)(H)—, —C(CH₃)(D)-, —CH₂CH₂CH₂—, —CH₂CH₂CH₂CH₂—, and —CH₂CH₂CH₂CH₂CH₂—.

The term “alkenylene,” as used herein, refers to a divalent group derived from a straight or branched chain hydrocarbon containing at least one carbon-carbon double bond.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The term “1,1-carbocyclylene” means a geminal divalent group derived from a cycloalkyl. A representative example is 1,1-C₃₋₆cycloalkylene (i.e.,

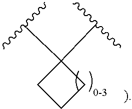

A further example is 1,1-cyclopropylene (i.e.,

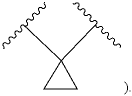 [SMILES: *C1(*)CC1]

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “difluoroalkyl,” as used herein, means an alkyl group, as defined herein, in which two hydrogen atoms are replaced by fluorine. Representative examples of difluoroalkyl include difluoromethyl and difluoroethyl.

The term “fluoroalkylene,” as used herein, means an alkylene group, as defined herein, in which one, two, three, four, five, six, seven or eight hydrogen atoms are replaced by fluorine. Representative examples of fluoroalkylene include, but are not limited to —CF₂—, —CH₂CF₂—, 1,2-difluoroethylene, 1,1,2,2-tetrafluoroethylene, 1,3,3,3-tetrafluoropropylene, 1,1,2,3,3-pentafluoropropylene, and perfluoropropylene such as 1,1,2,2,3,3-hexafluoropropylene.

The term “fluoroalkoxy,” as used herein, means at least one fluoroalkyl group, as defined herein, is appended to the parent molecular moiety through an oxygen atom. Representative examples of fluoroalkoxy include, but are not limited to, difluoromethoxy, trifluoromethoxy and 2,2,2-trifluoroethoxy.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,4-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, tetrahydrothiopyranyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane (e.g., 7- to 12-membered fused bicyclic heterocyclyl ring system such as hexahydro-2H-cyclopenta[b]furanyl, octahydro-3aH-cyclohepta[b]furanyl, or 3-oxabicyclo[3.1.0]hexanyl), or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group (e.g., a 7- to 12-membered spiro heterocyclyl ring system such as 2-oxaspiro[3.3]heptanyl, 3-oxaspiro[5.5]undecanyl, 6-oxaspiro[2.5]octanyl, or 5-oxaspiro[2.4]heptanyl), or a bridged heterocycle ring system in which two non-adjacent atoms of the ring are linked by an alkylene bridge of 1, 2, 3, or 4 carbon atoms (e.g., a 6- to 10-membered bridged bicyclic heterocyclyl ring system such as 7-oxabicyclo[2.2.1]heptanyl or 2-oxabicyclo[2.1.1]hexanyl), or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with a permitted valence of the atoms and the substituents, such that the selections and substitutions result in a stable compound, e.g., which does not spontaneously undergo transformation such as by rearrangement, cyclization, elimination, etc.

For the recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

### 2. Compounds

In one aspect, the invention provides compounds of formula (I), wherein R¹, R², R³, and n are as defined herein.

Unsubstituted or substituted rings (i.e., optionally substituted) such as aryl, heteroaryl, etc. are composed of both a ring system and the ring system's optional substituents. Accordingly, the ring system may be defined independently of its substituents, such that redefining only the ring system leaves any previous optional substituents present. For example, a 5- to 12-membered heteroaryl with optional substituents may be further defined by specifying the ring system of the 5- to 12-membered heteroaryl is a 5- to 6-membered heteroaryl (i.e., 5- to 6-membered heteroaryl ring system), in which case the optional substituents of the 5- to 12-membered heteroaryl are still present on the 5- to 6-membered heteroaryl, unless otherwise expressly indicated.

Where heterocyclic and heteroaromatic ring systems are defined to “contain” or as “containing” specified heteroatoms (e.g., 1-3 heteroatoms independently selected from the group consisting of O, N, and S), any ring atoms of the heterocyclic and heteroaromatic ring systems that are not one of the specified heteroatoms are carbon atoms.

In the following, numbered embodiments of the invention are disclosed. The first embodiment is denoted E1, and subsequent embodiments are denoted E1.1, E1.2, E1.3, E1.4, E1.5, E2, E3, etc.

E1. A compound of formula (I):

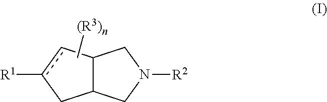

or a pharmaceutically acceptable salt thereof, wherein:
“ [symbol] ” represents a single bond or a double bond;
R¹ is

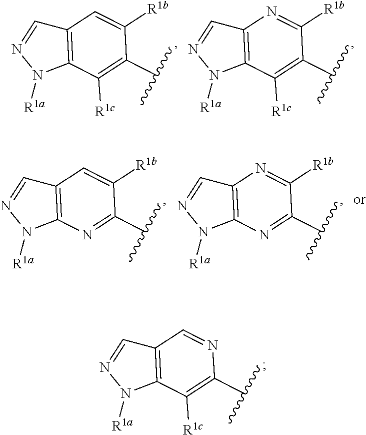

R^{1a} is G¹, -L¹-G¹, —C₂₋₄alkylene-OR^{A}, —C₂₋₄alkylene-NR^{A}R^{B}, C₁₋₆alkyl, C₁₋₆fluoroalkyl, or H;
R^{A} is G¹, -L¹-G¹, C₁₋₆alkyl, C₁₋₆fluoroalkyl, C₃₋₆cycloalkyl, or —C₁₋₃alkylene-C₃₋₆cycloalkyl;
R^{B} is H, C₁₋₆alkyl, C₁₋₆fluoroalkyl, C₃₋₆cycloalkyl, or —C₁₋₃alkylene-C₃₋₆cycloalkyl;
R^{1b} is halogen, C₁₋₄alkyl, C₁₋₂fluoroalkyl, CN, C₃₋₄cycloalkyl, —OC₁₋₃alkyl, —OC₁₋₂fluoroalkyl, or H;
R^{1c} is H, halogen, C₁₋₄alkyl, C₁₋₂fluoroalkyl, CN, phenyl, C₃₋₄cycloalkyl, —OR^{1d}, or —N(R^{1d})₂;
R^{1d}, at each occurrence, is independently H, C₁₋₄alkyl, C₁₋₂fluoroalkyl, C₃₋₄cycloalkyl, or —C₁₋₃alkylene-C₃₋₄cycloalkyl, wherein alternatively two R^{1d}, together with a nitrogen to which the two R^{1d} attach form a 4- to 6-membered heterocyclic ring optionally substituted with 1-4 substituents independently selected from the group consisting of halogen and C₁₋₄alkyl;
L¹ is C₁₋₅alkylene or C₂₋₅alkenylene;
G¹ is a 5- to 6-membered aromatic or partially unsaturated heterocyclic ring containing a first nitrogen and optionally 1-2 additional heteroatoms that are independently nitrogen, oxygen, or sulfur, the heterocyclic ring being attached at an unsaturated carbon in the heterocyclic ring or G¹ is phenyl, G¹ being optionally substituted with a first substituent selected from the group consisting of halogen, C₁₋₄alkyl, cyano, C₁₋₂fluoroalkyl, oxo, —OR¹⁰, —N(R¹⁰)₂, —C₁₋₃alkylene-OR¹⁰, C₃₋₅cycloalkyl, and —C₁₋₃alkylene-C₃₋₅cycloalkyl, and optionally further substituted with 1-2 substituents independently selected from the group consisting of halogen, C₁₋₄alkyl, cyano, and C₁₋₂fluoroalkyl;
R¹⁰, at each occurrence, is independently H, C₁₋₄alkyl, C₁₋₂fluoroalkyl, C₃₋₄cycloalkyl, or —C₁₋₃alkylene-C₃₋₄cycloalkyl, wherein alternatively two R¹⁰, together with a nitrogen to which the two R¹⁰ attach form a 4- to 6-membered heterocyclic ring optionally substituted with 1-4 substituents independently selected from the group consisting of halogen and C₁₋₄alkyl;
R² is G², -L²-G², -L³-C₁₋₆alkyl, —C₂₋₆alkylene-R^{2a}, C₁₋₆alkyl, C₁₋₆fluoroalkyl, or H;
L² is C₁₋₃alkylene or L³;
L³ is C(O), SO₂, S(O)(NH), C(O)NH, or C(O)O;
G² is a 4- to 12-membered heterocyclyl, a C₃₋₁₂carbocyclyl, a 5- to 12-membered heteroaryl, or a 6- to 12-membered aryl, wherein G² is optionally substituted with a first substituent selected from the group consisting of halogen, cyano, C₁₋₄alkyl, C₁₋₂fluoroalkyl, G^{2a}, oxo, —OR¹³, —N(R¹³)₂, —C₁₋₃alkylene-OR¹³, —C₁₋₃alkylene-N(R¹³)₂, —C(O)N(R¹³)₂, —C(O)OR¹³, —SO₂R¹³, and S(O)(NH)R¹³, optionally further substituted with oxo, and optionally further substituted with 1-3 substituents independently selected from the group consisting of halogen, cyano, C₁₋₄alkyl, and C₁₋₂fluoroalkyl;
G^{2a} is a C₃₋₄cycloalkyl;
R³, at each occurrence, is independently H, C₁₋₄alkyl, C₁₋₂fluoroalkyl, C₃₋₄cycloalkyl, or —C₁₋₃alkylene-C₃₋₄cycloalkyl, wherein alternatively two R¹³, together with a nitrogen to which the two R¹³ attach form a 4- to 6-membered heterocyclic ring optionally substituted with 1-4 substituents independently selected from the group consisting of halogen and C₁₋₄alkyl;
R^{2a} is —OR¹⁴, —N(R¹⁴)₂, —SO₂R¹⁴, S(O)(NH)R¹⁴, —NR¹⁴C(O)N(R¹⁴)₂, or —NR¹⁴C(O)OR¹⁴;
R¹⁴, at each occurrence, is independently H, C₁₋₄alkyl, C₁₋₂fluoroalkyl, C₃₋₄cycloalkyl, or —C₁-3alkylene-C₃₋₄cycloalkyl, wherein alternatively two R¹⁴, together with a nitrogen to which the two R¹⁴ attach form a 4- to 6-membered heterocyclic ring optionally substituted with 1-4 substituents independently selected from the group consisting of halogen and C₁₋₄alkyl;
R³, at each occurrence, is independently fluoro or C₁₋₄alkyl; and
n is 0, 1, or 2.

E1.1. The compound of E1, or a pharmaceutically acceptable salt thereof, wherein the compound has formula (II):

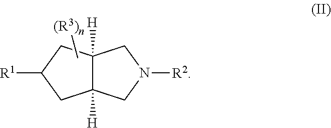

E1.2. The compound of E1 or E1.1, or a pharmaceutically acceptable salt thereof, wherein the compound has formula (II-A):

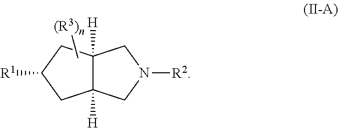

E1.3. The compound of E1 or E1.1, or a pharmaceutically acceptable salt thereof, wherein the compound has formula (II-B):

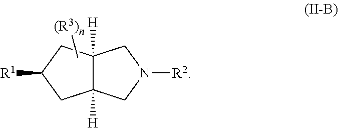

E1.4. The compound of E1, or a pharmaceutically acceptable salt thereof, wherein the compound has formula (III-A):

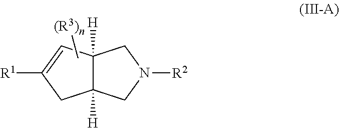

E1.5. The compound of E1, or a pharmaceutically acceptable salt thereof, wherein the compound has formula (III-B):

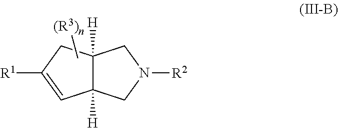

E2. The compound of any of E1-E1.5, or a pharmaceutically acceptable salt thereof, wherein R^{1a} is G¹; and G¹ is the optionally substituted 5- to 6-membered aromatic heterocyclic ring.

E3. The compound of any of E1-E2, or a pharmaceutically acceptable salt thereof, wherein the ring system of the optionally substituted 5- to 6-membered aromatic heterocyclic ring at G¹ contains 1-2 nitrogens.

E3.1. The compound of any of E1-E3, or a pharmaceutically acceptable salt thereof, wherein the ring system of the optionally substituted 5- to 6-membered aromatic heterocyclic ring at G¹ is pyridinyl or pyrazolyl.

E3.2. The compound of any of E1-E3.1, or a pharmaceutically acceptable salt thereof, wherein the ring system of the optionally substituted 5- to 6-membered aromatic heterocyclic ring at G¹ is pyridin-4-yl or pyrazol-4-yl.

E3.3. The compound of any of E1-E3.2, or a pharmaceutically acceptable salt thereof, wherein the ring system of the optionally substituted 5- to 6-membered aromatic heterocyclic ring at G¹ is pyrazol-4-yl.

E4. The compound of any of E1-E3.3, or a pharmaceutically acceptable salt thereof, wherein G¹ is optionally substituted with a first substituent selected from the group consisting of halogen, C₁₋₄alkyl, cyano, C₁₋₂fluoroalkyl, —OR¹⁰, C₃₋₅cycloalkyl, and —C₁₋₃alkylene-C₃₋₅cycloalkyl, and further optionally substituted with 1-2 substituents independently selected from the group consisting of halogen and C₁₋₄alkyl.

E4.1. The compound of any of E1-E4, or a pharmaceutically acceptable salt thereof, wherein G¹ is substituted with a first substituent selected from the group consisting of C₁₋₄alkyl and —OC₁₋₄alkyl.

E4.2. The compound of any of E1-E4.1, or a pharmaceutically acceptable salt thereof, wherein G¹ is substituted with a first substituent selected from the group consisting of methyl and —OCH₃.

E4.3. The compound of E4.1, or a pharmaceutically acceptable salt thereof, wherein G¹ is substituted with a first substituent that is C₁₋₄alkyl.

E4.4. The compound of E4.2 or E4.3, or a pharmaceutically acceptable salt thereof, wherein G¹ is substituted with a first substituent that is methyl.

E4.5. The compound of any of E1-E4.1 or E4.3, or a pharmaceutically acceptable salt thereof, wherein G¹ is

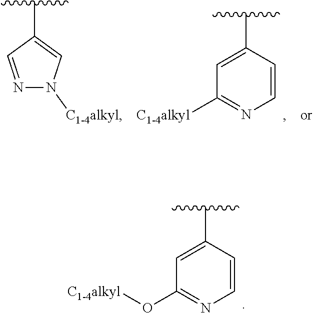

E4.6. The compound of any of E1-E4.5, or a pharmaceutically acceptable salt thereof, wherein G¹ is

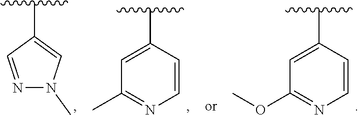

E4.7. The compound of E4.5, or a pharmaceutically acceptable salt thereof, wherein G¹ is

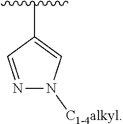

E4.8. The compound of E4.6 or E4.7, or a pharmaceutically acceptable salt thereof, wherein G¹ is

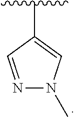 [SMILES: *c1cnn(C)c1]

E5. The compound of any of E1-E1.5, or a pharmaceutically acceptable salt thereof, wherein R^{1a} is G¹; and G¹ is the optionally substituted 5- to 6-membered partially unsaturated heterocyclic ring.

E6. The compound of any of E1-E1.5 or E5, or a pharmaceutically acceptable salt thereof, wherein the ring system of the optionally substituted 5- to 6-membered partially unsaturated heterocyclic ring at G¹ is 1,2-dihydropyridin-4-yl.

E6.1. The compound of any of E1-E1.5 or E5-E6, or a pharmaceutically acceptable salt thereof, wherein G¹ is optionally substituted with oxo.

E6.2. The compound of any of E1-E1.5 or E5-E6.1, or a pharmaceutically acceptable salt thereof, wherein G¹ is

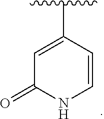 [SMILES: *c1cc[nH]c(=O)c1]

E7. The compound of any of E1-E6.2, or a pharmaceutically acceptable salt thereof, wherein G¹ is

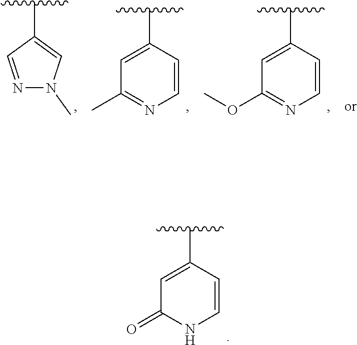

E8. The compound of any of E1-E7, or a pharmaceutically acceptable salt thereof, wherein R^{1b} is halogen or C₁₋₄alkyl.

E8.1. The compound of any of E1-E8, or a pharmaceutically acceptable salt thereof, wherein R^{1b} is chloro or methyl.

E8.2. The compound of E8, or a pharmaceutically acceptable salt thereof, wherein Rib is C₁₋₄alkyl.

E8.3. The compound of any of E8.1-E8.2, or a pharmaceutically acceptable salt thereof, wherein R^{1b} is methyl.

E9. The compound of any of E1-E8.3, or a pharmaceutically acceptable salt thereof, wherein R^{1c} is H.

E10. The compound of any of E1-E9, or a pharmaceutically acceptable salt thereof, wherein R² is G².

E11. The compound of any of E1-E9, or a pharmaceutically acceptable salt thereof, wherein R² is -L²-G².

E12. The compound of any of E1-E9 or E11, or a pharmaceutically acceptable salt thereof, wherein L² is C₁₋₃alkylene.

E13. The compound of any of E1-E9 or E11, or a pharmaceutically acceptable salt thereof, wherein L² is L³ and L³ is C(O).

E14. The compound of any of E1-E13, or a pharmaceutically acceptable salt thereof, wherein G² is the optionally substituted 4- to 12-membered heterocyclyl.

E15. The compound of any of E1-E14, or a pharmaceutically acceptable salt thereof, wherein the ring system of the optionally substituted 4- to 12-membered heterocyclyl at G² is a 4- to 8-membered heterocyclyl or an 8- to 10-membered fused bicyclic heterocyclyl, the heterocyclyls containing 1-2 heteroatoms independently selected from the group consisting of oxygen, nitrogen, and sulfur.

E15.1. The compound of E15, or a pharmaceutically acceptable salt thereof, wherein the ring system of the optionally substituted 4- to 12-membered heterocyclyl at G² is a 4- to 8-membered heterocyclyl ring system (i.e., G² is an optionally substituted 4- to 8-membered heterocyclyl).

E15.2. The compound of E15 or E15.1, or a pharmaceutically acceptable salt thereof, wherein the 4- to 8-membered heterocyclyl ring system at G² is a 4- to 8-membered monocyclic heterocyclyl ring system.

E15.3. The compound of E15.2, or a pharmaceutically acceptable salt thereof, wherein the 4- to 8-membered monocyclic heterocyclyl ring system at G² is a 4- to 6-membered monocyclic heterocyclyl ring system.

E15.4. The compound of E15.3, or a pharmaceutically acceptable salt thereof, wherein the ring system of the 4- to 6-membered monocyclic heterocyclyl ring system at G² is oxetanyl, tetrahydrofuranyl, tetrahydropyranyl, tetrahydrothiophenyl, tetrahydrothiopyranyl, or piperidinyl.

E15.5. The compound of E15.4, or a pharmaceutically acceptable salt thereof, wherein the ring system of the 4- to 6-membered monocyclic heterocyclyl ring system at G² is oxetan-3-yl, tetrahydrofuran-3-yl, tetrahydropyran-3-yl, tetrahydropyran-4-yl, tetrahydrothiopyran-3-yl, tetrahydrothiopyran-4-yl, or piperidin-3-yl.

E15.6. The compound of any of E1-E15.5, or a pharmaceutically acceptable salt thereof, wherein G² is optionally substituted with a first substituent selected from the group consisting of C₁₋₄alkyl, oxo, and —C(O)OR¹³, optionally further substituted with oxo, and optionally further substituted with 1-3 C₁₋₄alkyl.

E15.7. The compound of E15.6, or a pharmaceutically acceptable salt thereof, wherein G² is optionally substituted with a first substituent selected from the group consisting of methyl, oxo, and —C(O)OCH₃, optionally further substituted with oxo, and optionally further substituted with 1-3 methyl.

E15.8. The compound of E15.7, or a pharmaceutically acceptable salt thereof, wherein G² is

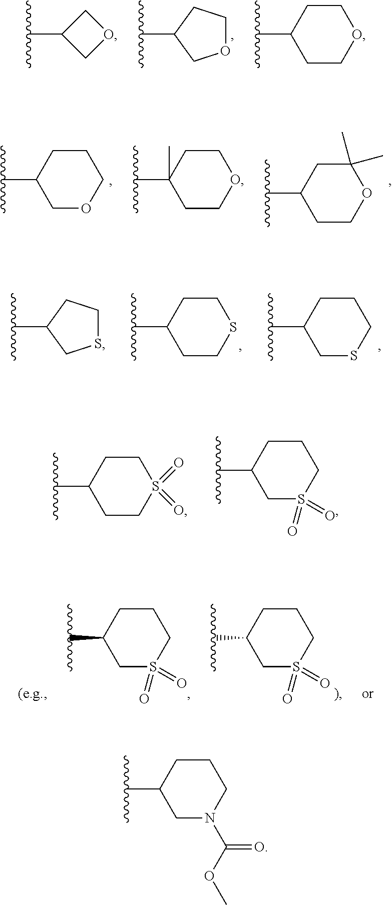

E15.9. The compound of E15.8, or a pharmaceutically acceptable salt thereof, wherein G² is

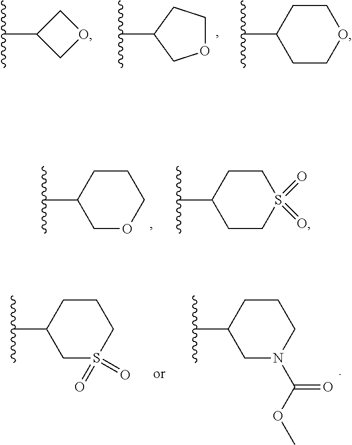

E15.10. The compound of E15, or a pharmaceutically acceptable salt thereof, wherein the ring system of the optionally substituted 4- to 12-membered heterocyclyl at G² is the 8- to 10-membered fused bicyclic heterocyclyl ring system (i.e., G² is an optionally substituted 8- to 10-membered fused bicyclic heterocyclyl).

E15.11. The compound of E15.10, or a pharmaceutically acceptable salt thereof, wherein the 8- to 10-membered fused bicyclic heterocyclyl ring system at G² is a 5- to 6-membered monocyclic heterocyclyl fused to a 6-membered arene, the 5- to 6-membered monocyclic heterocyclyl containing a sulfur, oxygen, or nitrogen atom.

E15.12. The compound of E15.11, or a pharmaceutically acceptable salt thereof, wherein the 8- to 10-membered fused bicyclic heterocyclyl ring system at G² is a 5- to 6-membered monocyclic heterocyclyl fused to a 6-membered arene, the 5- to 6-membered monocyclic heterocyclyl containing a sulfur atom.

E15.13. The compound of E15.12, or a pharmaceutically acceptable salt thereof, wherein G² is

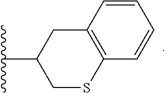 [SMILES: *C1CSc2ccccc2C1]

E16. The compound of any of E1-E13, or a pharmaceutically acceptable salt thereof, wherein G² is the optionally substituted C₃₋₁₂carbocyclyl.

E17. The compound of any of E1-E13 or E16, or a pharmaceutically acceptable salt thereof, wherein the ring system of the optionally substituted C₃₋₁₂carbocyclyl at G² is a C₃-6cycloalkyl.

E17.1. The compound of E17, or a pharmaceutically acceptable salt thereof, wherein the C₃₋₆cycloalkyl ring system at G² is a cyclohexyl ring system.

E17.2. The compound of any of E1-E13 or E16-E17.1, or a pharmaceutically acceptable salt thereof, wherein G² is optionally substituted with a first substituent selected from the group consisting of cyano, G^{2a}, —OR¹³, and —SO₂R¹³, and optionally further substituted with cyano.

E17.3. The compound of E17.2, or a pharmaceutically acceptable salt thereof, wherein G² is

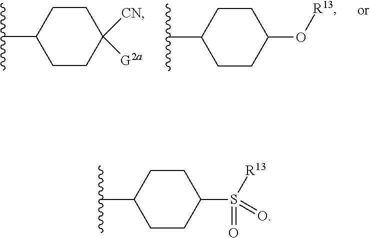

E17.4. The compound of any of E1-E17.3, or a pharmaceutically acceptable salt thereof, wherein R¹³ is C₁₋₄alkyl.

E17.5. The compound of E17.4, or a pharmaceutically acceptable salt thereof, wherein R¹³ is methyl.

E17.6. The compound of any of E1-E17.5, or a pharmaceutically acceptable salt thereof, wherein G^{2a} is cyclopropyl.

E17.7. The compound of any of E17-E17.6, or a pharmaceutically acceptable salt thereof, wherein G² is

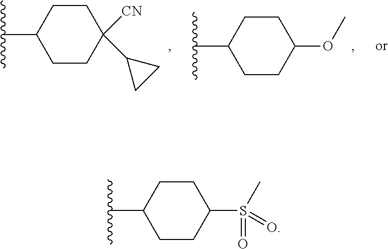

E18. The compound of any of E1-E13, or a pharmaceutically acceptable salt thereof, wherein G² is the optionally substituted 5- to 12-membered heteroaryl.

E19. The compound of any of E1-E13 or E18, or a pharmaceutically acceptable salt thereof, wherein the ring system of the optionally substituted 5- to 12-membered heteroaryl at G² is a 5- to 6-membered heteroaryl containing 1-3 heteroatoms independently selected from the group consisting of oxygen, nitrogen, and sulfur.

E19.1. The compound of any of E1-E13 or E18-E19, or a pharmaceutically acceptable salt thereof, wherein the ring system of the optionally substituted 5- to 12-membered heteroaryl at G² is pyrazolyl, thiazolyl, or thiadiazolyl.

E19.2. The compound of E19.1, or a pharmaceutically acceptable salt thereof, wherein the ring system of the optionally substituted 5- to 12-membered heteroaryl at G² is pyrazol-4-yl, thiazol-4-yl, or thiadiazol-5-yl.

E19.3. The compound of any of E1-E13 or E18-E19.1, or a pharmaceutically acceptable salt thereof, wherein the ring system of the optionally substituted 5- to 12-membered heteroaryl at G² is thiadiazolyl.

E19.4. The compound of E19.2 or E19.3, or a pharmaceutically acceptable salt thereof, wherein the ring system of the optionally substituted 5- to 12-membered heteroaryl at G is thiadiazol-5-yl.

E19.5. The compound of any of E1-E13 or E18-E19.4, or a pharmaceutically acceptable salt thereof, wherein G² is optionally substituted with a first substituent that is C₁₋₄alkyl, and optionally further substituted with 1-2 C₁₋₄alkyl.

E19.6. The compound of E19.5, or a pharmaceutically acceptable salt thereof, wherein G² is optionally substituted with a first substituent that is methyl, and optionally further substituted with 1-2 methyl.

E19.7. The compound of E19.5, or a pharmaceutically acceptable salt thereof, wherein G² is

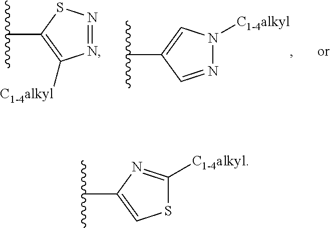

E19.8. The compound of E19.6 or E19.7, or a pharmaceutically acceptable salt thereof, wherein G² is

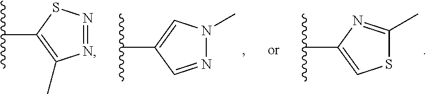

E19.9. The compound of E19.7, or a pharmaceutically acceptable salt thereof, wherein G² is

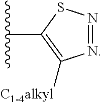

E19.10. The compound of any of E19.6-E19.9, or a pharmaceutically acceptable salt thereof, wherein G² is

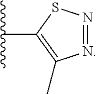 [SMILES: *c1snnc1C]

E20. The compound of any of E1-E13, or a pharmaceutically acceptable salt thereof, wherein G² is the optionally substituted 6- to 12-membered aryl.

E21. The compound of any of E1-E19.10, or a pharmaceutically acceptable salt thereof, wherein G² is

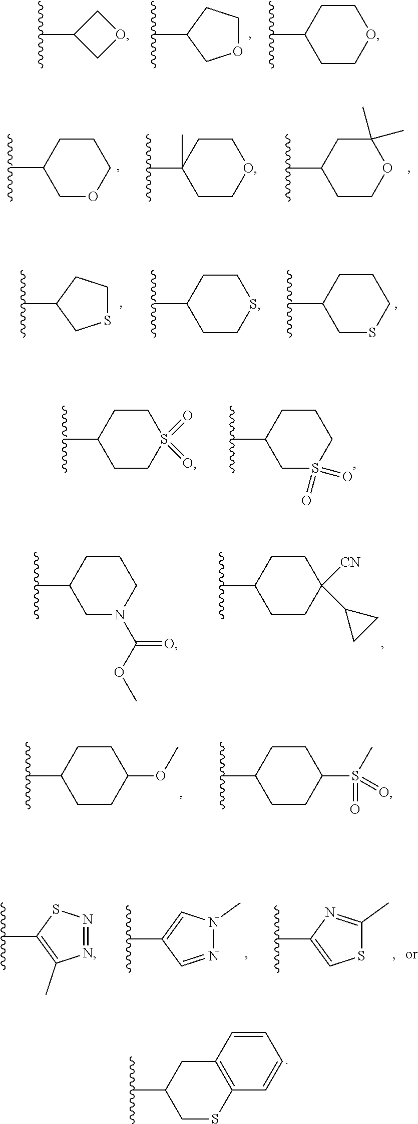

E22. The compound of any of E1-E21, or a pharmaceutically acceptable salt thereof, wherein n is 0.

E23. The compound of any of E1-E22, or a pharmaceutically acceptable salt thereof, wherein R¹ is

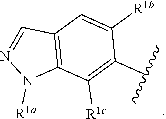

E24. The compound of any of E1-E23, or a pharmaceutically acceptable salt thereof, wherein the compound has formula (II-C):

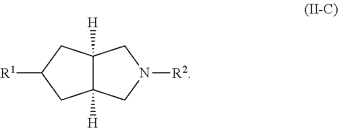

E24.1. The compound of E24, or a pharmaceutically acceptable salt thereof, wherein the compound has formula (II-C1):

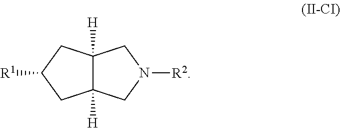

E24.2. The compound of E24, or a pharmaceutically acceptable salt thereof, wherein the compound has formula (II-C2):

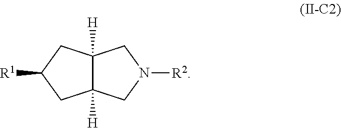

E25. The compound of any of E1-E23, or a pharmaceutically acceptable salt thereof, wherein the compound has formula (III-C):

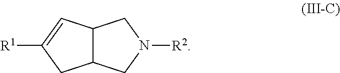

E25.1. The compound of E25, or a pharmaceutically acceptable salt thereof, wherein the compound has formula (III-C1):

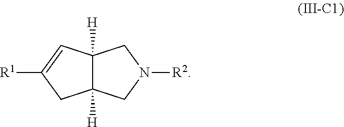

E25.2. The compound of E25, or a pharmaceutically acceptable salt thereof, wherein the compound has formula (III-C2):

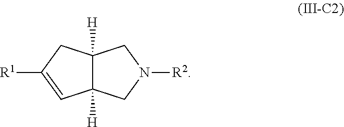

E26. The compound of E1 selected from the group consisting of:
6-((3aR,6aS)-1,2,3,3a,4,6a-hexahydrocyclopenta[c]pyrrol-5-yl)-5-methyl-1-(1-methyl-1H-pyrazol-4-yl)-1H-indazole;
tert-butyl (3aR,6aS)-5-(5-methyl-1-(1-methyl-1H-pyrazol-4-yl)-1H-indazol-6-yl)-3,3a,4,6a-tetrahydrocyclopenta[c]pyrrole-2(1H)-carboxylate;
5-methyl-1-(1-methyl-1H-pyrazol-4-yl)-6-((3aR,6aS)-2-(tetrahydro-2H-pyran-4-yl)-1,2,3,3a,4,6a-hexahydrocyclopenta[c]pyrrol-5-yl)-1H-indazole;
4-((3aR,6aS)-5-(5-methyl-1-(1-methyl-1H-pyrazol-4-yl)-1H-indazol-6-yl)-3,3a,4,6a-tetrahydrocyclopenta[c]pyrrol-2(1H)-yl)tetrahydro-2H-thiopyran 1,1-dioxide;
5-methyl-1-(1-methyl-1H-pyrazol-4-yl)-6-((3aR,5s,6aS)-2-(tetrahydro-2H-pyran-4-yl)octahydrocyclopenta[c]pyrrol-5-yl)-1H-indazole;
5-methyl-1-(1-methyl-1H-pyrazol-4-yl)-6-((3aR,5r,6aS)-2-(tetrahydro-2H-pyran-4-yl)octahydrocyclopenta[c]pyrrol-5-yl)-1H-indazole;
4-((3aR,5s,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide;
4-((3aR,5r,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide;
(4-methyl-1,2,3-thiadiazol-5-yl)((3aR,5s,6aS)-5-(5-methyl-1-(1-methyl-1H-pyrazol-4-yl)-1H-indazol-6-yl)hexahydrocyclopenta[c]pyrrol-2(1H)-yl)methanone;
(4-methyl-1,2,3-thiadiazol-5-yl)((3aR,5r,6aS)-5-(5-methyl-1-(1-methyl-1H-pyrazol-4-yl)-1H-indazol-6-yl)hexahydrocyclopenta[c]pyrrol-2(1H)-yl)methanone;
5-methyl-1-(1-methyl-1H-pyrazol-4-yl)-6-((3aR,5s,6aS)-2-(tetrahydro-2H-pyran-3-yl)octahydrocyclopenta[c]pyrrol-5-yl)-1H-indazole;
5-methyl-1-(1-methyl-1H-pyrazol-4-yl)-6-((3aR,5r,6aS)-2-(tetrahydro-2H-pyran-3-yl)octahydrocyclopenta[c]pyrrol-5-yl)-1H-indazole;
5-methyl-1-(1-methyl-1H-pyrazol-4-yl)-6-((3aR,5s,6aS)-2-(tetrahydrofuran-3-yl)octahydrocyclopenta[c]pyrrol-5-yl)-1H-indazole;
5-methyl-1-(1-methyl-1H-pyrazol-4-yl)-6-((3aR,5r,6aS)-2-(tetrahydrofuran-3-yl)octahydrocyclopenta[c]pyrrol-5-yl)-1H-indazole;
3-((3aR,5s,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide;
5-methyl-1-(1-methyl-1H-pyrazol-4-yl)-6-((3aR,5s,6aS)-2-(oxetan-3-yl)octahydrocyclopenta[c]pyrrol-5-yl)-1H-indazole;
5-methyl-1-(1-methyl-1H-pyrazol-4-yl)-6-((3aR,5r,6aS)-2-(oxetan-3-yl)octahydrocyclopenta[c]pyrrol-5-yl)-1H-indazole;
methyl 3-((3aR,5s,6aS)-5-(5-methyl-1-(1-methyl-1H-pyrazol-4-yl)-1H-indazol-6-yl)hexahydrocyclopenta[c]pyrrol-2(1H)-yl)piperidine-1-carboxylate;
methyl 3-((3aR,5r,6aS)-5-(5-methyl-1-(1-methyl-1H-pyrazol-4-yl)-1H-indazol-6-yl)hexahydrocyclopenta[c]pyrrol-2(1H)-yl)piperidine-1-carboxylate;
3-((3aR,5r,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide;
   or a pharmaceutically acceptable salt thereof.

E27. A pharmaceutical composition comprising the compound of any of E1-E26, or a pharmaceutically acceptable salt thereof, and a pharmaceutically acceptable carrier.

E28. A method of treating a disease or disorder comprising administering to a subject in need thereof a therapeutically effective amount of the compound of any of E1-E26, or a pharmaceutically acceptable salt thereof, or the pharmaceutical composition of E27, wherein the disease or disorder is a CNS disease or disorder, an immunological disease or disorder, a cancer, or leprosy.

E29. The method of E28, wherein the disease or disorder is a CNS disease or disorder.

E29.1. The method of E29, wherein the CNS disease or disorder is selected from the group consisting of familial/genetic and/or sporadic Parkinson's disease, a tauopathy, Alzheimer's disease, neuroinflammation, HIV-induced dementia, ALS, ischemic stroke, traumatic brain injury and spinal cord injury.

E29.2. The method of E29.1, wherein the CNS disease or disorder is familial/genetic and/or sporadic Parkinson's disease.

E30. The method of E28, wherein the disease or disorder is an immunological disease or disorder.

E30.1. The method of E30, wherein the immunological disease or disorder is selected from the group consisting of lymphomas, leukemias, multiple sclerosis, rheumatoid arthritis, systemic lupus erythematosus, autoimmune hemolytic anemia, pure red cell aplasia, idiopathic thrombocytopenic pupura (ITP), Evans Syndrome, vasculitis, bullous skin disorder, type I diabetes mellitus, Sjogren's syndrome, Delvic's disease, inflammatory myopathies, ankylosing spondylitis, and Crohn's disease.

E31. The method of E28, wherein the disease or disorder is a cancer.

E31.1. The method of E31, wherein the cancer is selected from the group consisting of renal cancer, breast cancer, lung cancer, prostate cancer, acute myelogenous leukemia (AML), papillary renal carcinoma, thyroid carcinoma, and low-grade glioma.

E32. The method of E28, wherein the disease or disorder is leprosy.

E33. A method of inhibiting LRRK2 in a subject comprising administering to the subject an amount effective to inhibit LRRK2, of the compound of any of E1-E26, or a pharmaceutically acceptable salt thereof, or the pharmaceutical composition of E27.

E33.1. The method of E33, wherein the subject suffers the disease or disorder of any of E28-E32.

E34. The method of E33 or E33.1, wherein LRRK2 is the G2019S mutant of LRRK2.

E35. The method of any of E28-E34, wherein the subject expresses the LRRK2 G2019S mutation.

E36. The method of any of E28-E35, wherein LRRK2 is amplified or overexpressed in the subject.

E37. A compound of any of E1-E26, or a pharmaceutically acceptable salt thereof, or the pharmaceutical composition of E27, for use in treating a disease or disorder selected from the group consisting of a CNS disease or disorder, an immunological disease or disorder, a cancer, and leprosy.

E38. Use of a compound of any of E1-E26, or a pharmaceutically acceptable salt thereof, or the pharmaceutical composition of E27, in the manufacture of a medicament for treating a disease or disorder selected from the group consisting of a CNS disease or disorder, an immunological disease or disorder, a cancer, and leprosy.

Compound names and/or structures can be assigned/determined by using the Struct=Name naming algorithm as part of CHEMDRAW® ULTRA.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

Compounds have a 3,3a,4,5,6,6a-hexahydro-1H-cyclopenta[c]pyrrole core structure that has a plane of symmetry as in the following two representative structures.

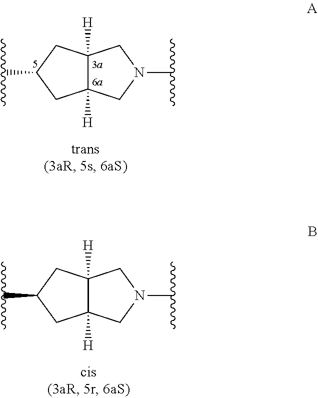 [SMILES: *[C@H]1C[C@@H]2CN(*)C[C@@H]2C1 ; *[C@@H]1C[C@@H]2CN(*)C[C@@H]2C1]

These structures are considered meso since A and B are superimposable with their respective mirror images. The 3a, 5, and 6a stereochemical designations are used herein for symmetrical structures of type A and B to designate relative stereochemistry between the ring fusion and the 5-position. Thus, when drawn in the orientation depicted above 3aR,5s,6aS refers to trans relative stereochemistry between the 5-position substituent and the ring fusion, and 3aR,5r,6aS refers to cis relative stereochemistry between the 5-position substituent and the ring fusion. The lower case s and r designations at the 5-position refer to pseudo asymmetry as described by G. P. Moss in “Basic terminology of stereochemistry (IUPAC Recommendations)” in Pure and Applied Chemistry (1996), 68 (12) 2193-2222. The person skilled in the art will understand that when structures A and B are drawn as the respective mirror images, chemical naming programs may, depending on the program, reverse the stereochemical designation for 3a and 6 positions from R to S and S to R, respectively, but that the pseudo asymmetry at the 5-position remains invariant, due to R having priority over S according to priority rules and the reversal of the carbons having R and S designations. Compounds of formula (I) or any of its subformulas may have a 5-position substituent in a trans configuration or a cis configuration, or may be prepared as a mixture of trans and cis.

It should be understood that the compound may possess tautomeric forms, as well as geometric isomers, and that these also constitute embodiments of the disclosure.

In the compounds of formula (I), and any subformulas, any “hydrogen” or “H,” whether explicitly recited or implicit in the structure, encompasses hydrogen isotopes ¹H (protium) and ²H (deuterium).

The present disclosure also includes isotopically-labeled compounds (e.g., deuterium labeled), where an atom in the isotopically-labeled compound is specified as a particular isotope of the atom. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F.

Isotopically-enriched forms of compounds of formula (I), or any subformulas, may generally be prepared by conventional techniques known to those skilled in the art or by processes analogous to those described in the accompanying Examples using an appropriate isotopically-enriched reagent in place of a non-isotopically-enriched reagent. The extent of isotopic enrichment can be characterized as a percent incorporation of a particular isotope at an isotopically-labeled atom (e.g., % deuterium incorporation at a deuterium label).

a. Pharmaceutically Acceptable Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

b. General Synthesis

Compounds of formula (I) or any of its subformulas may be prepared by synthetic processes or by metabolic processes. Preparation of the compounds by metabolic processes includes those occurring in the human or animal body (in vivo) or processes occurring in vitro.

Abbreviations: AcOH is acetic acid; BMS is borane dimethyl sulfide complex; Boc is tert-butyloxycarbonyl; BrettPhos-Pd-G3 is [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (CAS Number 1470372-59-8); t-BuXPhos is 2-di-tert-butylphosphino-2′,4′,6′-triisopropylbiphenyl; DAST is diethylaminosulfur trifluoride; DCE is 1,2-dichloroethane; DCM is dichloromethane; DIAD is diispropylazodicarboxylate; DIBAL is diisobutylaluminum hydride; DIEA and DIPEA both refer to N,N-diisopropylethylamine; DMF is N,N-dimethylformamide; EtOH is ethanol; Et₃SiCl is chlorotriethylsilane; HATU is 2-(7-aza-1H-benzotriazole-1-yl)-1,1,3,3-tetramethyluronium hexafluorophosphate; HOBt is hydroxybenzotriazole; LiAlH(OtBu)₃ is lithium tri-tert-butoxyaluminum hydride; LiHMDS is lithium bis(trimethylsilyl)amide; m-CPBA is meta-chloroperoxybenzoic acid; MeOH is methanol; MsCl is methanesulfonyl chloride; NaBH(OAc)₃ and STAB both refer to sodium triacetoxyborohydride; rt or r.t. is room temperature; NMP is N-methyl-2-pyrrolidone; Pd(dppf)Cl₂ is [1,1′-Bis(diphenylphosphino)ferrocene]dichloropalladium(II); Pd₂(dba)₃ is tris(dibenzylideneacetone)dipalladium(0); PPh₃ is triphenylphosphine; RuPhos-Pd-G3 is (2-dicyclohexylphosphino-2′,6′-diisopropoxy-1,1′-biphenyl)[2-(2′-amino-1,1′-biphenyl)]palladium(II) methanesulfonate (CAS Number 1445085-77-7); Selectfluor™ is 1-chloromethyl-4-fluoro-1,4-diazoniabicyclo[2.2.2]octane bis(tetrafluoroborate); t-BuOH is tert-butyl alcohol; t-BuOK is potassium tert-butoxide; TBAI is tetrabutylammonium iodide; THE is tetrahydrofuran; and TosMIC is toluenesulfonylmethyl isocyanide.

Compounds of formula (I) or any of its subformulas may be synthesized as shown in the following schemes.

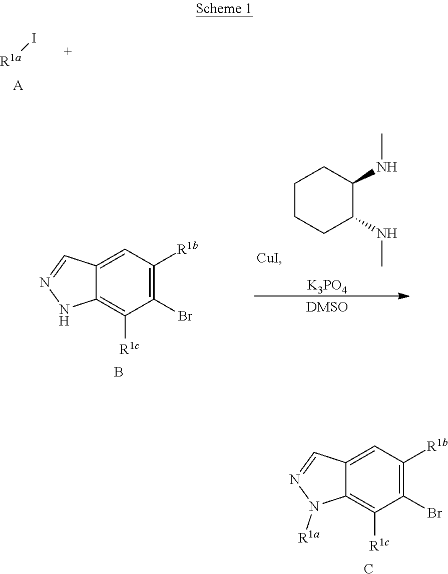

As shown in Scheme 1, iodide A may be coupled with 1H-indazole B under suitable Ullmann coupling reaction conditions to provide N-substituted indazole C.

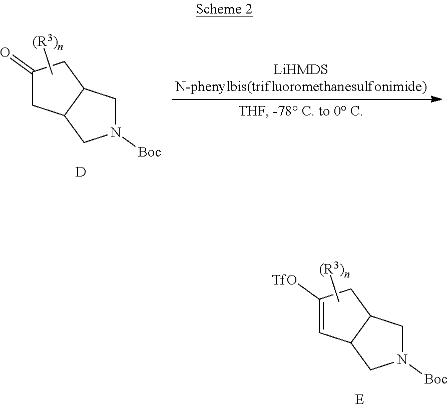

Scheme 2 illustrates the conversion of ketone D to enol triflate E by reaction with base (e.g., LiHMDS) and triflating reagent (e.g., N-phenylbis(trifluoromethanesulfonimide).

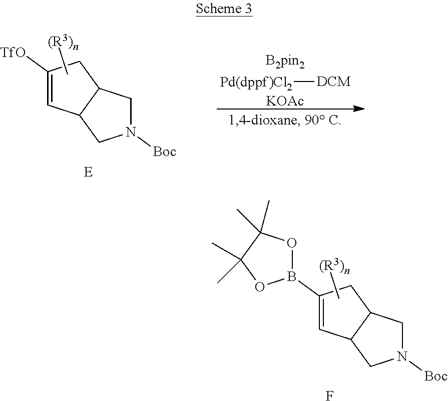

Scheme 3 illustrates the conversion of enol triflate E to pinacolborane F by coupling enol triflate E with Bis(pinacolato)diboron (B₂Pin₂) under suitable Miyaura borylation reaction conditions.

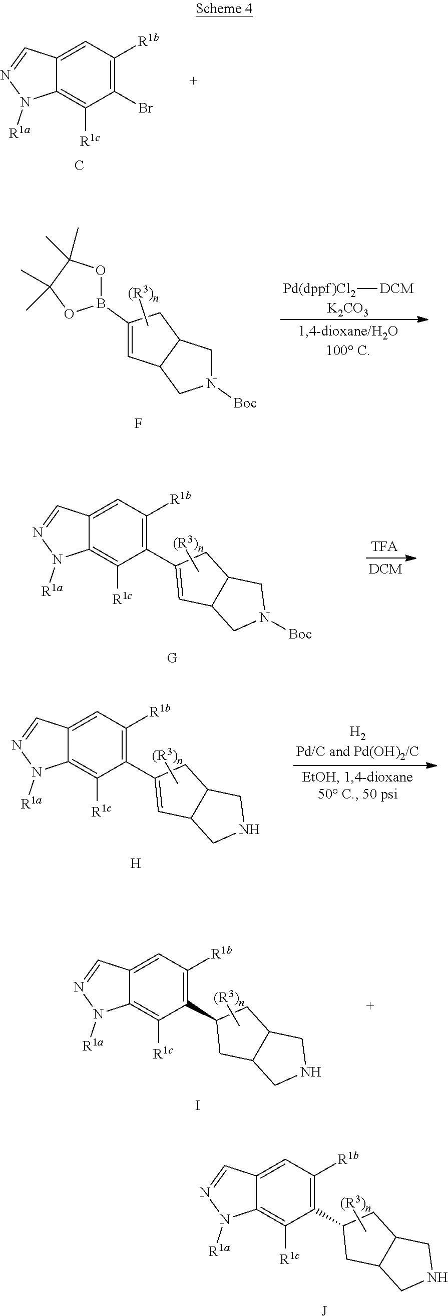

Scheme 4 illustrates a process for transforming intermediates C and F to products G to J. Intermediate C may be coupled with pinacolborane F under suitable Suzuki reaction conditions to provide Boc-protected G, which may be deprotected to provide deprotected H, which may be hydrogenated over a palladium catalyst to provide products I and J.

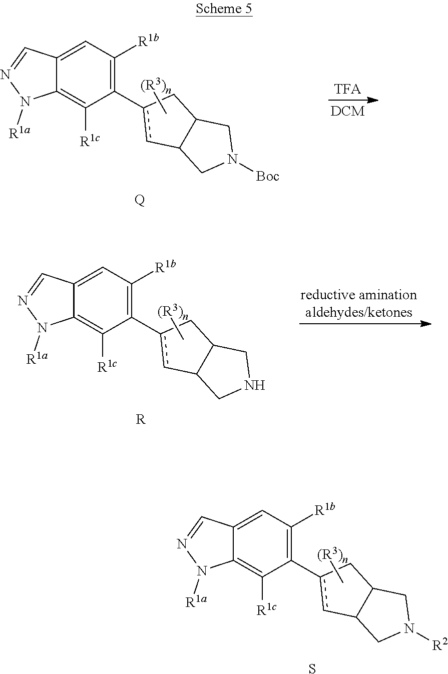

Scheme 5 illustrates a process for deprotecting compounds Q and reductive amination of the resultant secondary amines R to provide tertiary amine compounds S.

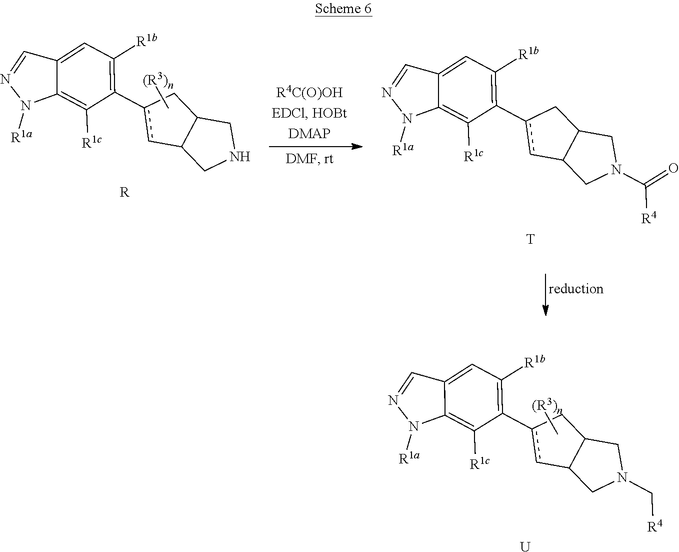

As shown in Scheme 6, compound R may be reacted with an appropriate carboxylic acid to form amide compound T, which may be reduced to generate compound U, wherein R⁴ is G², —C₁₋₂alkylene-G², —C₁₋₅alkylene-R^{2a}, or C₂₋₆alkyl, wherein G² and R^{2a} are as defined herein. Amide coupling conditions are well known in the art and include treating the reactants with a coupling agent such as HATU, in the presence of a base (e.g., DIPEA) in a solvent such as DMF or DCM. Amide reduction conditions are well known in the art and include treating the amide substrate with a reducing agent like DIBAL in DCM or LiAlH₄ in THF. The reaction may be conducted anywhere from −78° C. to room temperature. Compound T may also be reacted with LiAlD₄ to introduce deuterium atoms in place of the carbonyl.

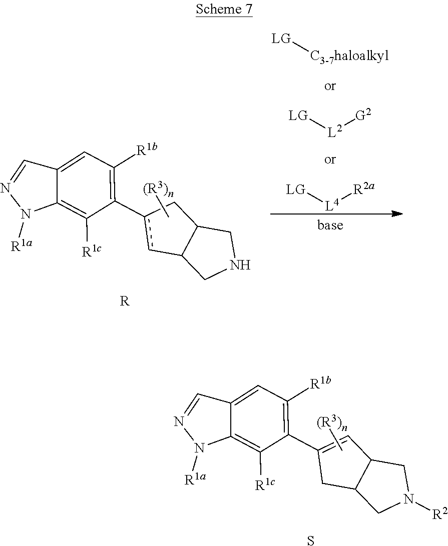

As shown in Scheme 7, compounds of formula R may be alkylated using standard secondary amine alkylation conditions to provide tertiary amines S, wherein R² is -L²-G², —C₂-6alkylene-R^{2a}, or C₃₋₇haloalkyl; L⁴ is a C₂₋₆alkylene group; LG is a leaving group (e.g., Cl, Br, I, mesylate, tosylate, triflate); and R^{2a}, L², and G² are as defined herein. An exemplary set of conditions for alkylation is to heat the reactants to about 70° C. in a solvent such as DMF or DMSO in the presence of a base such as Cs₂CO₃. Another exemplary set of alkylation conditions is to heat the reactants to about >100° C. in a sealed vessel in a microwave reactor using a solvent such as acetonitrile, DMF or DMSO in the presence of a tertiary amine base such as DIPEA.

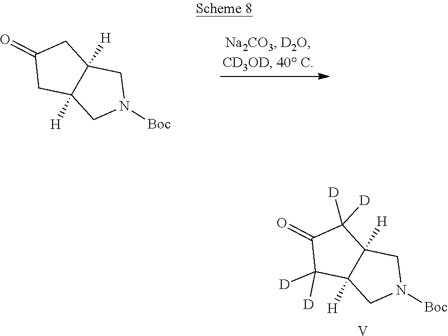

Scheme 8 shows a process to prepare intermediate V. Compound V may be processed according to Schemes 2-7 to arrive at compounds of the invention.

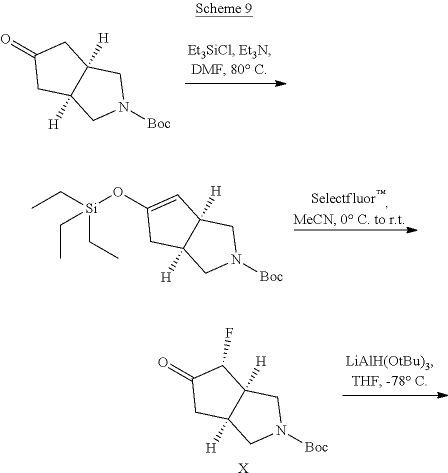

Scheme 9 shows a process to prepare intermediate X, in racemic form. Compound X may be processed according to Schemes 2-7 to arrive at compounds of the invention.

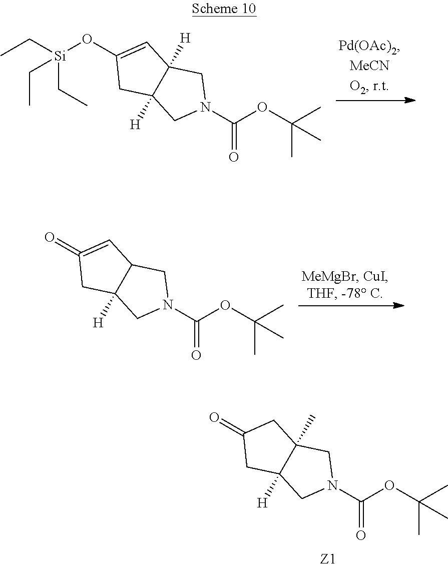

Scheme 10 shows a process to prepare intermediate Z1, in racemic form. Compound Z1 may be processed according to Schemes 2-7 to arrive at compounds of the invention.

Reductive amination conditions suitable for use in the processes of the above schemes are well known in the art. Representative reaction conditions for aldehyde reductive amination include treating the reactants with NaBH(OAc)₃ in solvents such as DCM, THF, and MeOH, and mixtures thereof, optionally in the presence of a base (e.g., DIPEA). Aldehyde reductive amination may also be effected by treatment with NaBH₃CN in EtOH with heating (e.g., to about 80° C.). Ketone reductive amination may be facilitated by addition of an acid like acetic acid to the solvent mixture (e.g., DCM-THF) and heating to 40° C. for about an hour. A representative solvent ratio of DCM:THF:AcOH is (3:3:0.5). Ketone reductive amination may also be effected by treatment with Ti(OiPr)₄ and NaBH₃CN or NaBH₄ in EtOH from room temperature to about 80° C. Ketone reductive amination may also be effected by treatment with 2-methylpyridine borane complex and acetic acid with heating to around 60° C. NaBD₃CN may be used instead of NaBH₃CN to incorporate deuterium and provide compounds enriched in deuterium over protium.

Ullmann coupling conditions suitable for use in the processes of Scheme 1 are well known in the art. Suitable Ullmann conditions include those generally outlined in Scheme 1, and as described in the Examples herein.

Miyaura borylation conditions suitable for use in the processes of Scheme 3 are well known in the art. Suitable Miyaura borylation conditions include those generally outlined in Scheme 3, and as described in the Examples herein.

Suzuki coupling conditions suitable for use in the processes of Scheme 4 are well known in the art. Suitable Suzuki conditions include those generally outlined in Scheme 4, and as described in the Examples herein.

Boronic acid or ester starting materials to prepare compounds of the invention may be purchased from commercial sources or prepared using procedures in the literature that are well known to those skilled in the art.

Schemes 1 and 4-7 showing processes for preparation of compounds where R¹ is

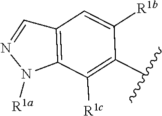

may also be adapted to the preparation of compounds where R¹ is

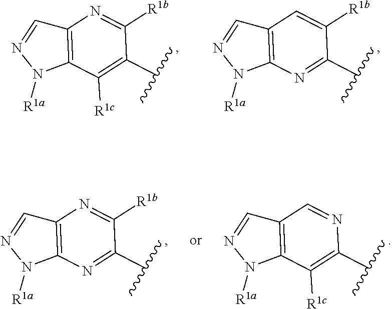

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### 3. Pharmaceutical Compositions and Formulations

The disclosed compounds may be incorporated into pharmaceutical compositions suitable for administration to a subject (such as a patient, which may be a human or non-human). The disclosed compounds may also be provided as formulations, such as spray-dried dispersion formulations.

The pharmaceutical compositions and formulations may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I) or any of its subformulas) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I) or any of its subformulas, may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methylparaben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I) or any of its subformulas) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I) or any of its subformulas), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol, and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I) or any of its subformulas), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

### 4. Methods of Use

The disclosed compounds, pharmaceutical compositions and formulations may be used in methods for the treatment of CNS disorders, such as familial/genetic and/or sporadic Parkinson's disease, tauopathies, or Alzheimer's disease. The disclosed compounds and pharmaceutical compositions may also be used in methods for decreasing LRRK2 activity in a mammal, including the G2019S mutant of LRRK2. The methods further include cotherapeutic methods for improving treatment outcomes. In the methods of use described herein, additional therapeutic agent(s) may be administered simultaneously, separately, or sequentially with the disclosed compounds and compositions.

a. Treating Disorders

The disclosed compounds, pharmaceutical compositions and formulations may be used in methods for treating, preventing, ameliorating, controlling, reducing, or reducing the risk of a variety of diseases or disorders, or symptoms of the diseases or disorders, in which a patient would benefit from inhibition of LRRK2. Diseases or disorders in which a patient would benefit from inhibition of LRRK2 may include CNS disorders, such as familial/genetic and/or sporadic Parkinson's disease, tauopathies, and Alzheimer's disease. The methods may comprise administering to a subject in need of such treatment a therapeutically effective amount of the compound of formula (I) or any of its subformulas or a pharmaceutically acceptable salt thereof, or a pharmaceutical composition comprising a therapeutically effective amount of a compound of formula (I) or any of its subformulas or a pharmaceutically acceptable salt thereof.

In some embodiments, the disclosure provides a method for treating a CNS disorder, such as familial/genetic and/or sporadic Parkinson's disease, tauopathies, and Alzheimer's disease, comprising administering to a subject in need thereof a therapeutically effective amount of the compound of formula (I) or any of its subformulas or a pharmaceutically acceptable salt thereof, or a pharmaceutical composition comprising a therapeutically effective amount of a compound of formula (I) or any of its subformulas or a pharmaceutically acceptable salt thereof.

LRRK2 pathogenic mutants have been associated with aspects of Parkinson's disease, including dopaminergic neuronal cell death, impaired dopamine neurotransmission and locomotive activity, defects in protein synthesis and degradation, inflammatory responses, and oxidative damage (BMB Rep 2015 May; 48(5):243-8). Treatment of Parkinson's disease may include: treatment of neurologic damage associated with Parkinson's disease; or treating or improving dopaminergic tone to provide symptomatic relief (e.g., in treating, alleviating, ameliorating, or managing motor and non-motor symptoms of Parkinson's disease). Motor symptoms of Parkinson's disease include bradykinesia, rigidity, and resting tremor. Non-motor symptoms include cognitive dysfunction, autonomic dysfunction, emotional changes and sleep disruption.

Additional diseases or disorders in which a patient may benefit from inhibition of LRRK2 include Alzheimer's disease, mild cognitive impairment, the transition from mild cognitive impairment to Alzheimer's disease, and tauopathy disorders characterized by hyperphosphorylation of tau.

Tauopathies are neurodegenerative disorders characterized by the deposition of abnormal tau protein in the brain, including disorders characterized by hyperphosphorylated of tau such as argyrophilic grain disease, Picks disease, corticobasal degeneration, progressive supranuclear palsy, inherited frontotemporal dementia and Parkinson's linked to chromosome 17.

Additional diseases or disorders in which a patient may benefit from inhibition of LRRK2 include neuroinflammation, including neuroinflammation associated with of microglial inflammatory responses associated with multiple sclerosis, HIV-induced dementia, ALS, ischemic stroke, traumatic brain injury and spinal cord injury.

Additional diseases or disorders in which a patient may benefit from inhibition of LRRK2 include diseases of the immune system including lymphomas, leukemias, multiple sclerosis, rheumatoid arthritis, systemic lupus erythematosus, autoimmune hemolytic anemia, pure red cell aplasia, idiopathic thrombocytopenic pupura (ITP), Evans Syndrome, vasculitis, bullous skin disorder, type I diabetes mellitus, Sjogren's syndrome, Delvic's disease, inflammatory myopathies, and ankylosing spondylitis.

Additional diseases or disorders in which a patient may benefit from inhibition of LRRK2 include renal cancer, breast cancer, lung cancer, prostate cancer, and acute myelogenous leukemia (AML) in subjects expressing the LRRK2 G2019S mutation.

Additional diseases or disorders in which a patient may benefit from inhibition of LRRK2 include papillary renal and thyroid carcinomas in a subject in whom LRRK2 is amplified or overexpressed.

An additional disease or disorder in which a patient may benefit from inhibition of LRRK2 is Low-Grade Glioma.

Additional diseases or disorders in which a patient may benefit from inhibition of LRRK2 include chronic autoimmune diseases including Crohn's disease and leprosy.

The compounds and compositions may be further useful in a method for the prevention, treatment, control, amelioration, or reduction of risk of the diseases, disorders and conditions noted herein. The compounds and compositions may be further useful in a method for the prevention, treatment, control, amelioration, or reduction of risk of the aforementioned diseases, disorders and conditions, in combination with other agents.

In the treatment of conditions such as those that would benefit from inhibition of LRRK2, an appropriate dosage level may be about 0.01 to 500 mg per kg patient body weight per day, which can be administered in single or multiple doses. The dosage level may be about 0.1 to about 250 mg/kg per day, or about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, or 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in some embodiments, the disclosure relates to a method for inhibiting LRRK2 in at least one cell, comprising the step of contacting the at least one cell with at least one disclosed compound or at least one product of a disclosed method in an amount effective to inhibit LRRK2 in the at least one cell. In some embodiments, the cell is mammalian, for example, human. In some embodiments, the cell has been isolated from a subject prior to the contacting step. In some embodiments, contacting is via administration to a subject.

In some embodiments, the invention relates to a method for inhibiting LRRK2 in a subject, comprising the step of administering to the subject at least one disclosed compound or at least one product of a disclosed method in a dosage and amount effective to inhibit LRRK2 in the subject. In some embodiments, the subject is mammalian, for example, human. In some embodiments, the mammal has been diagnosed with a need for LRRK2 inhibition prior to the administering step. In some embodiments, the mammal has been diagnosed with a need for LRRK2 inhibition prior to the administering step. In some embodiments, the method further comprises the step of identifying a subject in need of LRRK2 inhibition.

b. Inhibition of LRRK2

In some embodiments, the disclosure relates to a method for inhibiting LRRK2 in a mammal, comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In some embodiments, the disclosure relates to a method for inhibiting the G2019S mutant LRRK2 in a mammal, comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In some embodiments, the compound administered inhibits LRRK2 or G2019S mutant with an IC₅₀ of less than about 10 μM, less than about 5 μM, less than about 1 μM, less than about 500 nM, or less than about 100 nM. In some embodiments, the compound administered inhibits LRRK2 or G2019S mutant with an IC₅₀ of between about 10 μM and about 1 nM, about 1 M and about 1 nM, about 100 nM and about 1 nM, or about 10 nM and about 1 nM.

In some embodiments, the mammal is a human. In some embodiments, the mammal has been diagnosed with a need for reduction of LRRK2 or G2019S mutant activity prior to the administering step. In some embodiments, the method further comprises the step of identifying a mammal in need of reducing LRRK2 or G2019S mutant activity. In some embodiments, the inhibition of LRRK2 or G2019S mutant treats a disorder associated with LRRK2 activity in the mammal.

In some embodiments, inhibition of LRRK2 in a mammal is associated with the treatment of a disorder associated with the G2019S mutant of LRRK2, such as a disorder disclosed herein.

In some embodiments, the disclosure provides a method for inhibiting LRRK2 or G2019S mutant in a cell, comprising the step of contacting the cell with an effective amount of at least one disclosed compound or a pharmaceutically acceptable salt thereof. In some embodiments, the cell is mammalian (e.g., human). In some embodiments, the cell has been isolated from a mammal prior to the contacting step. In some embodiments, contacting is via administration to a mammal.

c. Combination Therapies

The disclosed compounds may be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone.

In one aspect, the compounds can be co-administered with anti-Alzheimer's agents, beta-secretase inhibitors, gamma-secretase inhibitors, muscarinic agonists, muscarinic potentiators HMG-CoA reductase inhibitors, NSAIDs and anti-amyloid antibodies. In a further aspect, the compounds can be administered in combination with sedatives, hypnotics, anxiolytics, antipsychotics, selective serotonin reuptake inhibitors (SSRIs), monoamine oxidase inhibitors (MAOIs), 5-HT2 antagonists, GlyT1 inhibitors and the like such as, but not limited to: risperidone, clozapine, haloperidol, fluoxetine, prazepam, xanomeline, lithium, phenobarbitol, and salts thereof and combinations thereof. In a further aspect, the subject compound may be used in combination with levodopa (with or without a selective extracerebral decarboxylase inhibitor), anticholinergics such as biperiden and M4 selective antagonists, COMT inhibitors such as entacapone, A2a adenosine antagonists, cholinergic agonists, NMDA receptor antagonists and dopamine agonists.

In some embodiments, the compounds of the invention can be employed in combination with other agents to increase their efficacy and/or safety. the compound can be administered in combination with (but not limited to) α-synuclein fibrillation and/or aggregation inhibitors, MAO-B inhibitors (e.g. Rasagiline, Selegiline), antiemetics, L-DOPA, dopamine agonist (e.g. Rotigotine, Lisuride, Pramipexole), an agent that increases extracellular dopamine levels (e.g., amphetamine, methylphenidate, or lisdexamfetamine), nicotinic receptor agonist (e.g. Amantadine), dopaminergic agonists (e.g. Cabergoline, Ropinirole, Quinpirole, Ropinirole, Pramipexole, Pergolide and Bromocriptine), DOPA decarboxylase inhibitors (e.g. Carbidopa and Benserazide), COMT inhibitors (e.g. Tolcapone and Entacapone), an adenosine A2a antagonists, mGlu4 modulators, mGlu5 modulators, or growth factors (e.g. brain derived neurotrophic factor (BDNF)).

In other embodiments, the compounds can be used in combination with other treatments, including but not limited to glycoprotein IIb/IIIa receptor inhibitors, ion channel blockers (e.g. calcium channel blockers), beta blockers, cyclooxygenase inhibitors, agents that improve lung and/or kidney function, angiotensin system inhibitor (e.g., angiotensin-converting enzyme inhibitors), renin inhibitors, PARP inhibitors, Src inhibitors, cardiovascular disorder related agents, anti-hypertension agents, hypercholesterolemia and type II diabetes related agents, anti-inflammatory agents, anti-thrombotic agents, fibrinolytic agents, anti-platelet agents, lipid reducing agents, thrombin inhibitors, apoptosis inhibitors, and/or substances that bind to cellular adhesion molecules and prevent white blood cells from attaching to them (e.g., polypeptides, polyclonal and monoclonal antibodies).

In the methods of use described herein, additional therapeutic agent(s) may be administered simultaneously, separately, or sequentially with the disclosed compounds and compositions. Sequential administration includes administration before or after the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula (I) or any of its subformulas. The above combinations include combinations of a compound of the present invention not only with one other active compound, but also with two or more other active compounds.

The disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound may be used. However, the combination therapy can also be administered on overlapping schedules. It is also envisioned that the combination of one or more active ingredients and a disclosed compound can be more efficacious than either as a single agent. Thus, when used in combination with one or more other active ingredients, the disclosed compounds and the other active ingredients can be used in lower doses than when each is used singly.

The pharmaceutical compositions and methods of the present invention can further comprise other therapeutically active compounds as noted herein which are usually applied in the treatment of the above mentioned pathological conditions.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefore, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the disclosed compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

d. Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kinds of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

### 5. Kits

In one aspect, the disclosure provides a kit comprising at least one disclosed compound or a pharmaceutically acceptable salt thereof, or a pharmaceutical composition comprising at least one disclosed compound or a pharmaceutically acceptable salt thereof and instructions for use thereof.

In some embodiments, the at least one disclosed compound and the at least one agent are co-formulated. In some embodiments, the at least one disclosed compound and the at least one agent are co-packaged. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

The kits may comprise information, instructions, or both that use of the kit will provide treatment for medical conditions in mammals (particularly humans). The information and instructions may be in the form of words, pictures, or both, and the like. In addition or in the alternative, the kit may include the compound, a composition, or both; and information, instructions, or both, regarding methods of application of compound, or of composition, preferably with the benefit of treating or preventing medical conditions in mammals (e.g., humans).

### 6. Examples

All NMR spectra were recorded on a 400 MHz AMX Bruker NMR spectrometer. ¹H chemical shifts are reported in 6 values in ppm downfield with the deuterated solvent as the internal standard. Data are reported as follows: chemical shift, multiplicity (s=singlet, bs=broad singlet, d=doublet, t=triplet, q=quartet, dd=doublet of doublets, m=multiplet, ABq=AB quartet), coupling constant, integration. Reversed-phase LCMS analysis was performed using an Agilent 1200 system comprised of a binary pump with degasser, high-performance autosampler, thermostatted column compartment, C18 column, diode-array detector (DAD) and an Agilent 6150 MSD with the following parameters. The gradient conditions were 5% to 95% acetonitrile with the aqueous phase 0.1% TFA in water over 1.4 minutes. Samples were separated on a Waters Acquity UPLC BEH C18 column (1.7 μm, 1.0×50 mm) at 0.5 mL/min, with column and solvent temperatures maintained at 55° C. The DAD was set to scan from 190 to 300 nm, and the signals used were 220 nm and 254 nm (both with a band width of 4 nm). The MS detector was configured with an electrospray ionization source, and the low-resolution mass spectra were acquired by scanning from 140 to 700 AMU with a step size of 0.2 AMU at 0.13 cycles/second, and peak width of 0.008 minutes. The drying gas flow was set to 13 liters per minute at 300° C. and the nebulizer pressure was set to 30 psi. The capillary needle voltage was set at 3000 V, and the fragmentor voltage was set at 100V. Data acquisition was performed with Agilent Chemstation and Analytical Studio Reviewer software.

### Abbreviations

AcOH is acetic acid;
aq is aqueous
BINAP is 2,2′-Bis(diphenylphosphino)-1,1′-binaphthyl;
Boc is tert-butyloxycarbonyl;
BrettPhos-Pd-G3 is [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (CAS Number 1470372-59-8);
tBuOH is tert-butyl alcohol;
B₂Pin₂ is Bis(pinacolato)diboron;
Celite® is diatomaceous earth;
DCE is 1,2-dichloroethane;
DCM is dichloromethane;
DIAD is diisopropylazodicarboxylate;
DIPEA is N,N-diisopropylethylamine;
DMAP is 4-dimethylaminopyridine
DMF is N,N-dimethylformamide;
DMSO is dimethylsulfoxide;
eq, eq., or equiv is equivalent(s);
Et₂O is diethylether;
EtOAc is ethyl acetate;
EtOH is ethanol;
Et₃N is triethylamine;
HATU is 2-(7-aza-1H-benzotriazole-1-yl)-1,1,3,3-tetramethyluronium hexafluorophosphate;
HOBt is hydroxybenzotriazole;
h or h. is hour(s);
hex is hexane;
IPA is isopropyl alcohol;
LCMS is liquid chromatography mass spectrometry;
LiAlD₄ is lithium aluminum deuteride;
LiAlH(OtBu)₃ is lithium tri-tert-butoxyaluminum hydride;
LiHMDS is lithium bis(trimethylsilyl)amide;
m-CPBA is meta-chloroperoxybenzoic acid;
MeCN is acetonitrile;
MeMgBr is methyl magnesium bromide;
MeOH is methanol;
MeOD is deuterated methanol;
min or min. is minute(s);
MTBE is methyl tert-butyl ether;
NaBH(OAc)₃ is sodium triacetoxyborohydride (i.e., STAB);
NMP is N-methyl-2-pyrrolidone;
Pd(OAc)₂ is palladium(II) acetate;
Pd(dppf)Cl₂ is [1,1′-Bis(diphenylphosphino)ferrocene]dichloropalladium(II);
PPh₃ is triphenylphosphine;
RP-HPLC is reverse phase high-performance liquid chromatography;
RuPhos-Pd-G3 is (2-dicyclohexylphosphino-2′,6′-diisopropoxy-1,1′-biphenyl)[2-(2′-amino-1,1′-biphenyl)]palladium(II) methanesulfonate (CAS Number 1445085-77-7);
rt, RT, or r.t. is room temperature;
sat. is saturated;
SFC is supercritical fluid chromatography;
soln. is solution;
TESCL is chlorotriethylsilane;
TFA is trifluoroacetic acid;
THF is tetrahydrofuran;
tosyl is toluenesulfonyl.
   b. Preparation of Intermediates

### Intermediate Example 1. 6-Bromo-5-methyl-1-(1-methyl-1H-pyrazol-4-yl)-1H-indazole

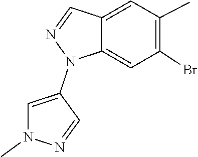

The title compound was synthesized similarly as previously described in WO2020/092136. To a solution of 6-bromo-5-methyl-1H-indazole (500 mg, 2.37 mmol, 1 eq), 1-methyl-4-iodopyrazole (739 mg, 3.55 mmol, 1.5 eq) in DMSO (5 mL), (rel, trans)-N,N-dimethylcyclohexane-1,2-diamine (0.19 mL, 1.18 mmol, 0.5 eq), CuI (90 mg, 0.47 mmol, 0.2 eq), and K₃PO₄ (1530 mg, 7.11 mmol, 3 eq) were added. The resulting mixture was stirred at 90° C. under N₂ for 5 h, cooled to rt, and diluted with H₂O (5 mL). The organics were then extracted with DCM (3×50 mL). The combined organic layers were dried over Na₂SO₄ and concentrated in vacuo to afford a crude residue. The crude residue was purified using flash chromatography on silica (0-100% EtOAc in hexanes) to provide the title compound (637 mg, 92%). LCMS ES-MS [M+H]⁺=291 and 293.

### Intermediate Example 2. (rac)-tert-Butyl (3aR,6aS)-5-(4,4,5,5-tetramethyl-1,3,2-dioxaborolan-2-yl)-3,3a,4,6a-tetrahydrocyclopenta[c]pyrrole-2(1H)-carboxylate

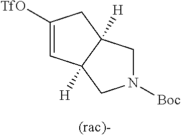 [SMILES: CC(C)(C)OC(=O)N1C[C@@H]2CC(OS(=O)(=O)C(F)(F)F)=C[C@@H]2C1]

Step A. (rac)-tert-Butyl (3aR,6aR)-5-(((trifluoromethyl)sulfonyl)oxy)-3,3a,4,6a-tetrahydrocyclopenta[c]pyrrole-2(1H)-carboxylate. To a solution of tert-butyl (3aR,6aS)-5-oxohexahydrocyclopenta[c]pyrrole-2(1H)-carboxylate (1000 mg, 4.4 mmol, 1 eq) in THE (12 mL) at −78° C., LiHMDS (1.0 M in THF, 4.9 mL, 4.9 mmol, 1.1 eq) was added dropwise under N₂. The reaction mixture was then stirred at −78° C. for 20 min, after which time, a solution of N-phenyl trifluoromethanesulfonimide (1665 mg, 4.7 mmol, 1.05 eq) in THF (12 mL) was added dropwise. The reaction mixture was then gradually warmed to 0° C. and stirred for 3 h at 0° C. Upon completion, the reaction mixture was quenched with a sat. aq. NH₄Cl (10 mL). The resulting mixture was then extracted with DCM (3×100 mL). The combined organics were dried over Na₂SO₄, filtered, and concentrated. The crude residue was purified using flash chromatography on silica (0-100% EtOAc in hexanes) to provide the title compound (613.2 mg, 38%). LCMS ES-MS [M+2H-tBu]⁺=302.

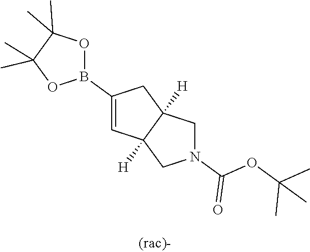 [SMILES: CC(C)(C)OC(=O)N1C[C@@H]2CC(B3OC(C)(C)C(C)(C)O3)=C[C@@H]2C1]

Step B. (rac)-tert-Butyl (3aR,6aS)-5-(4,4,5,5-tetramethyl-1,3,2-dioxaborolan-2-yl)-3,3a,4,6a-tetrahydrocyclopenta[c]pyrrole-2(1H)-carboxylate. To a mixture of (rac)-tert-butyl (3aR,6aR)-5-(((trifluoromethyl)sulfonyl)oxy)-3,3a,4,6a-tetrahydrocyclopenta[c]pyrrole-2(1H)-carboxylate (613.2 mg, 1.72 mmol, 1 eq), Bis(pinacolato)diboron (523 mg, 2.06 mmol, 1.2 eq), KOAc (539 mg, 5.49 mmol, 3.2 eq) and Pd(dppf)Cl₂-DCM (140 mg, 0.17 mmol, 0.1 eq), 1,4-dioxane (10 mL) was added. The resulting mixture was purged with N₂ and stirred at 90° C. overnight. The reaction was then filtered through a pad of Celite® and concentrated to provide the crude mixture of the title compound, which was used for the next step without purification (575 mg, assumed theoretical yield). LCMS ES-MS [M+2H-tBu]⁺=280.

### Intermediate Example 3. 5-Methyl-1-(1-methyl-1H-pyrazol-4-yl)-6-((3aR,5s,6aS)-octahydrocyclopenta[c]pyrrol-5-yl)-1H-indazole and 5-methyl-1-(1-methyl-1H-pyrazol-4-yl)-6-((3aR,5r,6aS)-octahydrocyclopenta[c]pyrrol-5-yl)-1H-indazole

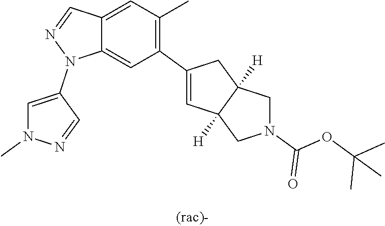 [SMILES: Cc1cc2cnn(-c3cnn(C)c3)c2cc1C1=C[C@@H]2CN(C(=O)OC(C)(C)C)C[C@@H]2C1]

Step A. (rac)-tert-Butyl (3aR,6aS)-5-(5-methyl-1-(1-methyl-1H-pyrazol-4-yl)-1H-indazol-6-yl)-3,3a,4,6a-tetrahydrocyclopenta[c]pyrrole-2(1H)-carboxylate. To a solution of (rac)-tert-butyl (3aR,6aS)-5-(4,4,5,5-tetramethyl-1,3,2-dioxaborolan-2-yl)-3,3a,4,6a-tetrahydrocyclopenta[c]pyrrole-2(1H)-carboxylate (575 mg, 1.72 mmol, 1 eq) in 1,4-dioxane (7.5 mL), 6-bromo-5-methyl-1-(1-methylpyrazol-4-yl)indazole (506 mg, 1.74 mmol, 1 eq), Pd(dppf)Cl₂-DCM (142 mg, 0.17 mmol, 0.1 eq), K₂CO₃ (731 mg, 5.22 mmol, 3 eq), and H₂O (1.5 mL) were added. The resulting mixture was then stirred at 100° C. overnight, cooled to rt, diluted with EtOAc (10 mL), filtered through a pad of Celite®, washed with EtOAc, and concentrated under reduced pressure. The crude residue was purified using flash chromatography on silica (0-100% EtOAc in hexanes) to provide the title compound (441.2 mg, 61%). ¹H NMR (400 MHz, CDCl₃) δ 8.00 (s, 1H), 7.82 (s, 1H), 7.74 (s, 1H), 7.52 (s, 1H), 7.28 (s, 1H), 5.65 (s, 1H), 3.99 (s, 3H), 3.72 (m, 1H), 3.61-3.43 (m, 3H), 3.18 (m, 1H), 2.99 (m, 2H), 2.51 (d, J=14.0 Hz, 1H), 2.41 (s, 3H), 1.46 (s, 9H); LCMS ES-MS [M+H]⁺=420.

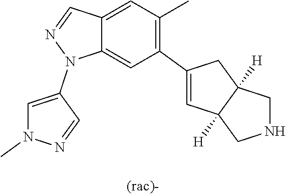 [SMILES: Cc1cc2cnn(-c3cnn(C)c3)c2cc1C1=C[C@@H]2CNC[C@@H]2C1]

Step B. (rac)-6-((3aR,6aS)-1,2,3,3a,4,6a-Hexahydrocyclopenta[c]pyrrol-5-yl)-5-methyl-1-(1-methyl-1H-pyrazol-4-yl)-1H-indazole. (rac)-tert-Butyl (3aR,6aS)-5-(5-methyl-1-(1-methyl-1H-pyrazol-4-yl)-1H-indazol-6-yl)-3,3a,4,6a-tetrahydrocyclopenta[c]pyrrole-2(1H)-carboxylate (506.4 mg, 1.21 mmol, 1 eq) was dissolved in DCM (4 mL), then TFA (2.3 mL, 30.2 mmol, 25 eq) was added. The reaction was stirred at rt for 6 h, diluted with H₂O (3 mL) and DCM (20 mL) and neutralized with K₂CO₃. The organics were then extracted with 3:1 CHCl₃:IPA (3×20 mL). The combined organics were dried over Na₂SO₄, filtered, and concentrated in vacuo. The crude product was used for the next step without further purification (385.5 mg, assumed theoretical yield). ¹H NMR (400 MHz, MeOD) δ 8.07 (s, 1H), 8.07 (s, 1H), 7.84 (s, 1H), 7.61 (s, 1H), 7.45 (s, 1H), 5.70 (q, J=2.1 Hz, 1H), 3.99 (s, 3H), 3.80 (tp, J=7.7, 2.4 Hz, 1H), 3.52 (dd, J=11.5, 8.1 Hz, 1H), 3.44 (dd, J=11.8, 7.7 Hz, 1H), 3.37-3.32 (m, 1H), 3.29-3.13 (m, 3H), 2.70-2.63 (m, 1H), 2.45 (s, 3H), LCMS ES-MS [M+H]⁺=320.

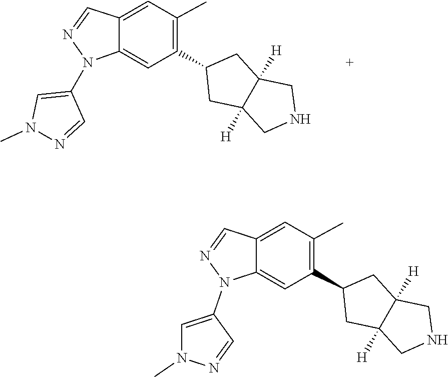

Step C. 5-Methyl-1-(1-methyl-1H-pyrazol-4-yl)-6-((3aR,5s,6aS)-octahydrocyclopenta[c]pyrrol-5-yl)-1H-indazole and 5-methyl-1-(1-methyl-1H-pyrazol-4-yl)-6-((3aR,5r,6aS)-octahydrocyclopenta[c]pyrrol-5-yl)-1H-indazole. To a solution of (rac)-6-((3aR,6aS)-1,2,3,3a,4,6a-hexahydrocyclopenta[c]pyrrol-5-yl)-5-methyl-1-(1-methyl-1H-pyrazol-4-yl)-1H-indazole (375.1 mg, 1.17 mmol, 1 eq) in EtOH (20 mL) and 1,4-dioxane (3 mL) were added 10% Pd/C (125 mg, 0.12 mmol, 0.1 eq) and 20% Pd(OH)₂/C (82.5 mg, 0.12 mmol, 0.1 eq). The reaction was charged with H₂ (50 psi) and stirred at 50° C. for 2 days in a Parr Shaker. The reaction mixture was then filtered and concentrated under reduced pressure to provide title compounds (377 mg, 99%). The crude product was used for the next step without further purification. LCMS ES-MS [M+H]⁺=322; [M+H]⁺=322.

d. Preparation of Representative Compounds

### Example 1. 4-((3aR,5s,6aS)-5-(5-Methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide and 4-((3aR,5r,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide (compound 7 and compound 8)

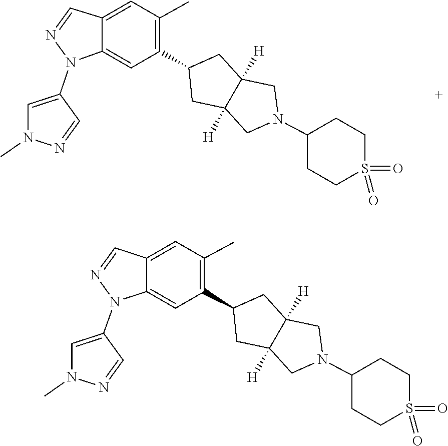

To a solution of 5-methyl-1-(1-methyl-1H-pyrazol-4-yl)-6-((3aR,5s,6aS)-octahydrocyclopenta[c]pyrrol-5-yl)-1H-indazole and 5-methyl-1-(1-methyl-1H-pyrazol-4-yl)-6-((3aR,5r,6aS)-octahydrocyclopenta[c]pyrrol-5-yl)-1H-indazole (20 mg total, 0.06 mmol, 1 eq) in THF (0.5 mL), N,N-diisopropylethylamine (16 μL, 0.09 mmol, 1.5 eq) was added followed by tetrahydro-4H-thiopyran-4-one 1,1-dioxide (18.4 mg, 0.12 mmol, 2 eq). This reaction mixture was stirred at 50° C. for 10 min. NaBH(OAc)₃ (40 mg, 0.19 mmol, 3 eq) was then added and the reaction was stirred at 50° C. for 6 h. The reaction mixture was then quenched with MeOH (1 mL) and concentrated under reduced pressure. The crude residue was purified using reverse-phase HPLC (8-95% CH₃CN in H₂O containing 0.05% NH₄OH) to afford the title compounds: 4-((3aR,5s,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide (4.8 mg, 17%) LCMS ES-MS [M+H]⁺=454 and 4-((3aR,5r,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide (13.8 mg, 48%). LCMS ES-MS [M+H]⁺=454.

### Example 2. (rac)-3-((3aR,5s,6aS)-5-(5-Methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide and (rac)-3-((3aR,5r,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide (Compound 15 and Compound 20)

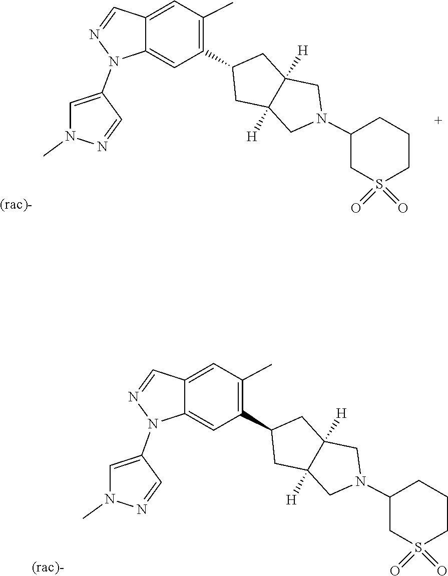 [SMILES: Cc1cc2cnn(-c3cnn(C)c3)c2cc1[C@H]1C[C@@H]2CN(C3CCCS(=O)(=O)C3)C[C@@H]2C1 ; Cc1cc2cnn(-c3cnn(C)c3)c2cc1[C@@H]1C[C@@H]2CN(C3CCCS(=O)(=O)C3)C[C@@H]2C1]

To a solution of 5-methyl-1-(1-methyl-1H-pyrazol-4-yl)-6-((3aR,5s,6aS)-octahydrocyclopenta[c]pyrrol-5-yl)-1H-indazole and 5-methyl-1-(1-methyl-1H-pyrazol-4-yl)-6-((3aR,5r,6aS)-octahydrocyclopenta[c]pyrrol-5-yl)-1H-indazole (50 mg total, 0.156 mmol, 1 eq) in MeOH (1.37 mL) and acetic acid (137 μL) was added dihydro-2H-thiopyran-3(4H)-one 1,1-dioxide (69 mg, 0.47 mmol, 3 eq) followed by 2-methylpyridine borane complex (85.4 mg, 0.80 mmol, 5.1 eq). The reaction mixture was purged with N₂ and gradually heated to 60° C. until the starting material was completely consumed (~16 h). Upon completion, the crude residue was purified using reverse-phase HPLC (5-80% CH₃CN in H₂O containing 0.05% NH₄OH) to afford the title compounds. (rac)-3-((3aR,5s,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide (10.2 mg, 14%). ¹H NMR (400 MHz, MeOD) δ 8.07 (s, 1H), 8.02 (d, J=0.8 Hz, 1H), 7.82 (d, J=0.5 Hz, 1H), 7.56 (s, 1H), 7.42 (s, 1H), 3.99 (s, 3H), 3.59-3.48 (m, 1H), 3.36 (m, 1H), 3.14-2.98 (m, 5H), 2.89-2.77 (m, 3H), 2.48 (s, 3H), 2.27 (ddd, J=23.1, 9.2, 6.2 Hz, 2H), 2.22-2.10 (m, 2H), 2.00-1.79 (m, 5H), 1.56-1.44 (m, 1H); LCMS ES-MS [M+H]⁺=454 and (rac)-3-((3aR,5r,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide (29.8 mg, 42%) LCMS ES-MS [M+H]⁺=454.

### Example 3. (4-Methyl-1,2,3-thiadiazol-5-yl)((3aR,5s,6aS)-5-(5-methyl-1-(1-methyl-1H-pyrazol-4-yl)-1H-indazol-6-yl)hexahydrocyclopenta[c]pyrrol-2(1H)-yl)methanone and (4-methyl-1,2,3-thiadiazol-5-yl)((3aR,5r,6aS)-5-(5-methyl-1-(1-methyl-1H-pyrazol-4-yl)-1H-indazol-6-yl)hexahydrocyclopenta[c]pyrrol-2(1H)-yl)methanone (compound 9 and compound 10)

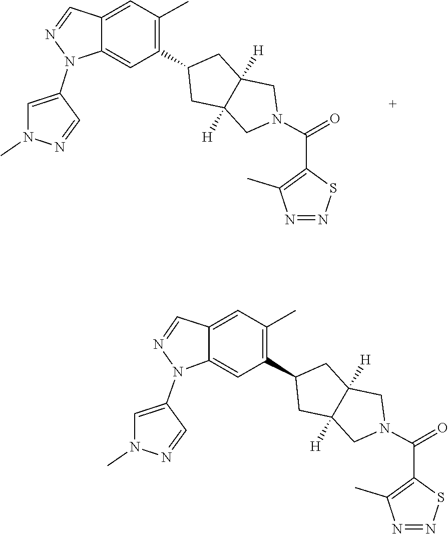

To a solution of 5-methyl-1-(1-methyl-1H-pyrazol-4-yl)-6-((3aR,5s,6aS)-octahydrocyclopenta[c]pyrrol-5-yl)-1H-indazole and 5-methyl-1-(1-methyl-1H-pyrazol-4-yl)-6-((3aR,5r,6aS)-octahydrocyclopenta[c]pyrrol-5-yl)-1H-indazole (20 mg total, 0.06 mmol, 1 eq) in DMF (1 mL), 1-(3-dimethylaminopropyl)-3-ethylcarbodiimide hydrochloride (18 mg, 0.09 mmol, 1.5 eq), 4-methyl-1,2,3-thiadiazole-5-carboxylic acid (13.5 mg, 0.09 mmol, 1.5 eq), DMAP (3.8 mg, 0.03 mmol, 0.5 eq), and HOBt (4.2 mg, 0.03 mmol, 0.5 eq) were added. The reaction mixture was then stirred at rt for 1.5 h. Upon completion, the reaction mixture was quenched with MeOH (1 mL) and concentrated. The crude residue was purified using reverse-phase HPLC (8-95% CH₃CN in H₂O containing 0.05% NH₄OH) to afford the title compounds: (4-methyl-1,2,3-thiadiazol-5-yl)((3aR,5s,6aS)-5-(5-methyl-1-(1-methyl-7H-pyrazol-4-yl)-1H-indazol-6-yl)hexahydrocyclopenta[c]pyrrol-2(1H)-yl)methanone (1.5 mg, 50 z). LCMS ES-MS [M+H]⁺=448 and (4-methyl-1,2,3-thiadiazol-5-yl)((3aR,5r,6aS)-5-(5-methyl-3-(1-methyl-H-pyrazol-4-yl)-1H-indazol-6-yl)hexahydrocyclopenta[c]pyrrol-2(1H)-yl)methanone (7.3 mg, 260.). LCMS ES-MS [M+H]⁺=448.

The compounds shown in Table 1 may be prepared similarly to the compound described above, with appropriate starting materials.

TABLE 1

| No. | STRUCTURE | NAME | ¹H-NMR and/or ES-MS [M + H]⁺ |
|---|---|---|---|
| 1 | 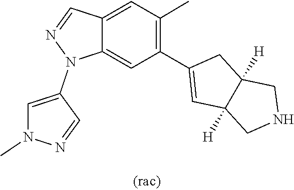 [SMILES: Cc1cc2cnn(-c3cnn(C)c3)c2cc1C1=C[C@@H]2CNC[C@@H]2C1] | (rac)-6-((3aR,6aS)- 1,2,3,3a,4,6a- hexahydrocyclopenta[c] pyrrol-5-yl)-5-methyl-1-(1- methyl-1H-pyrazol-4-yl)- 1H-indazole | ¹H NMR (400 MHz, MeOD) δ 8.07 (s, 1H), 8.07 (s, 1H), 7.84 (s, 1H), 7.61 (s, 1H), 7.45 (s, 1H), 5.70 (q, J = 2.1 Hz, 1H), 3.99 (s, 3H), 3.80 (tp, J = 7.7, 2.4 Hz, 1H), 3.52 (dd, J = 11.5, 8.1 Hz, 1H), 3.44 (dd, J = 11.8, 7.7 Hz, 1H), 3.37-3.32 (m, 1H), 3.29-3.13 (m, 3H), 2.70- 2.63 (m, 1H), 2.45 (s, 3H); [M + H]⁺ = 320 |
| 2 | 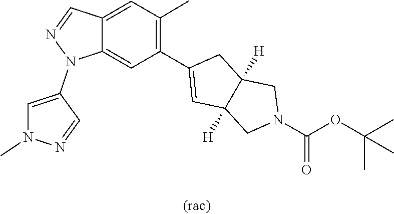 [SMILES: Cc1cc2cnn(-c3cnn(C)c3)c2cc1C1=C[C@@H]2CN(C(=O)OC(C)(C)C)C[C@@H]2C1] | (rac)-tert-butyl (3aR,6aS)- 5-(5-methyl-1-(1-methyl- 1H-pyrazol-4-yl)-1H- indazol-6-yl)-3,3a,4,6a- tetrahydrocyclopenta[c] pyrrole-2(1H)-carboxylate | [M + H]⁺ = 420 |
| 3 | 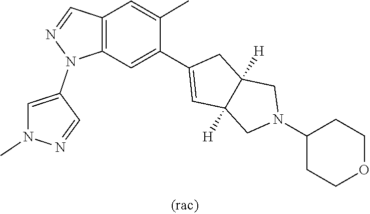 [SMILES: Cc1cc2cnn(-c3cnn(C)c3)c2cc1C1=C[C@@H]2CN(C3CCOCC3)C[C@@H]2C1] | (rac)-5-methyl-1-(1- methyl-1H-pyrazol-4-yl)- 6-((3aR,6aS)-2- (tetrahydro-2H-pyran-4- yl)-1,2,3,3a,4,6a- hexahydrocyclopenta[c] pyrrol-5-yl)-1H-indazole | ¹H NMR (400 MHz, MeOD) δ 8.08 (s, 1H), 8.05 (d, J = 0.7 Hz, 1H), 7.84 (s, 1H), 7.59 (s, 1H), 7.43 (s, 1H), 5.66 (q, J = 1.9 Hz, 1H), 3.99 (s, 3H), 3.95 (dd, J = 11.5, 2.9 Hz, 2H), 3.56-3.47 (m, 1H), 3.41 (td, J = 11.9, 2.2 Hz, 2H), 3.06-2.95 (m, 4H), 2.59-2.49 (m, 1H), 2.48- 2.36 (m, 2H), 2.43 (s, 3H), 2.25 (m, 1H), 1.91-1.81 (m, 2H), 1.63- 1.49 (m, 2H); [M + H]⁺ = 404 |
| 4 | 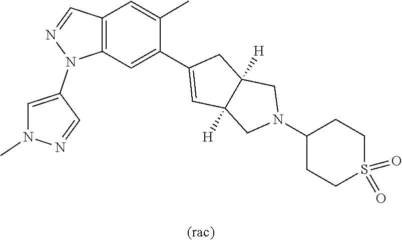 [SMILES: Cc1cc2cnn(-c3cnn(C)c3)c2cc1C1=C[C@@H]2CN(C3CCS(=O)(=O)CC3)C[C@@H]2C1] | (rac)-4-((3aR,6aS)-5-(5- methyl-1-(1-methyl-1H- pyrazol-4-yl)-1H-indazol- 6-yl)-3,3a,4,6a- tetrahydrocyclopenta[c] pyrrol-2(1H)-yl)tetrahydro- 2H-thiopyran 1,1-dioxide | [M + H]⁺ = 452 |
| 5 | 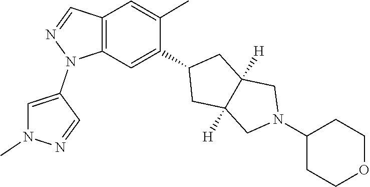 [SMILES: Cc1cc2cnn(-c3cnn(C)c3)c2cc1[C@H]1C[C@@H]2CN(C3CCOCC3)C[C@@H]2C1] | 5-methyl-1-(1-methyl-1H- pyrazol-4-yl)-6- ((3aR,5s,6aS)-2- (tetrahydro-2H-pyran-4- yl)octahydrocyclopenta[c] pyrrol-5-yl)-1H-indazole | ¹H NMR (400 MHz, MeOD) δ 8.08 (s, 1H), 8.03 (d, J = 0.8 Hz, 1H), 7.83 (d, J = 0.5 Hz, 1H), 7.58 (s, 1H), 7.46 (s, 1H), 4.00 (s, 3H), 4.00- 3.94 (m, 2H), 3.58-3.48 (m, 1H), 3.43 (td, J = 11.9, 2.2 Hz, 2H), 3.30-3.24 (m, 2H), 2.91-2.80 (m, 2H), 2.49 (s, 3H), 2.29 (m, 1H), 2.06 (dd, J = 9.9, 7.5 Hz, 2H), 1.93- 1.81 (m, 6H), 1.55 (qd, J = 13.1, 12.6, 4.6 Hz, 2H); [M + H]⁺ = 406 |
| 6 | 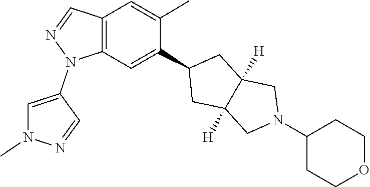 [SMILES: Cc1cc2cnn(-c3cnn(C)c3)c2cc1[C@@H]1C[C@@H]2CN(C3CCOCC3)C[C@@H]2C1] | 5-methyl-1-(1-methyl-1H- pyrazol-4-yl)-6- ((3aR,5r,6aS)-2- (tetrahydro-2H-pyran-4- yl)octahydrocyclopenta[c] pyrrol-5-yl)-1H-indazole | [M + H]⁺ = 406 |
| 7 | 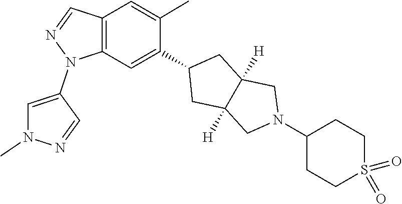 [SMILES: Cc1cc2cnn(-c3cnn(C)c3)c2cc1[C@H]1C[C@@H]2CN(C3CCS(=O)(=O)CC3)C[C@@H]2C1] | 4-((3aR,5s,6aS)-5-(5- methyl-1-(1-methyl-1H- pyrazol-4-yl)-1H-indazol-6- yl)hexahydrocyclopenta[c] pyrrol-2(1H)- yl)tetrahydro-2H- thiopyran 1,1-dioxide | [M + H]⁺ = 454 |
| 8 | 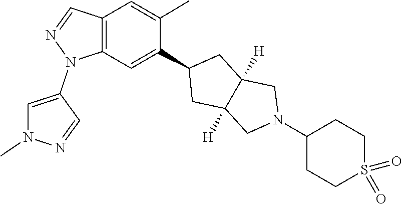 [SMILES: Cc1cc2cnn(-c3cnn(C)c3)c2cc1[C@@H]1C[C@@H]2CN(C3CCS(=O)(=O)CC3)C[C@@H]2C1] | 4-((3aR,5r,6aS)-5-(5- methyl-1-(1-methyl-1H- pyrazol-4-yl)-1H-indazol-6- yl)hexahydrocyclopenta[c] pyrrol-2(1H)- yl)tetrahydro-2H- thiopyran 1,1-dioxide | [M + H]⁺ = 454 |
| 9 | 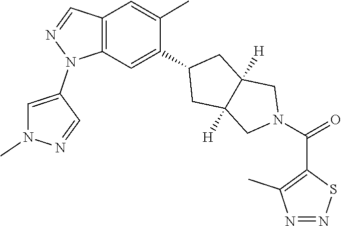 [SMILES: Cc1cc2cnn(-c3cnn(C)c3)c2cc1[C@H]1C[C@@H]2CN(C(=O)c3snnc3C)C[C@@H]2C1] | (4-methyl-1,2,3- thiadiazol-5- yl)((3aR,5s,6aS)-5-(5- methyl-1-(1-methyl-1H- pyrazol-4-yl)-1H-indazol-6- yl)hexahydrocyclopenta[c] pyrrol-2(1H)- yl)methanone | [M + H]⁺ = 448 |
| 10 | 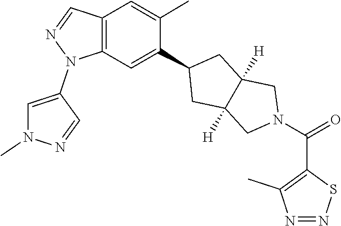 [SMILES: Cc1cc2cnn(-c3cnn(C)c3)c2cc1[C@@H]1C[C@@H]2CN(C(=O)c3snnc3C)C[C@@H]2C1] | (4-methyl-1,2,3- thiadiazol-5- yl)((3aR,5r,6aS)-5-(5- methyl-1-(1-methyl-1H- pyrazol-4-yl)-1H-indazol-6- yl)hexahydrocyclopenta[c] pyrrol-2(1H)- yl)methanone | [M + H]⁺ = 448 |
| 11 | 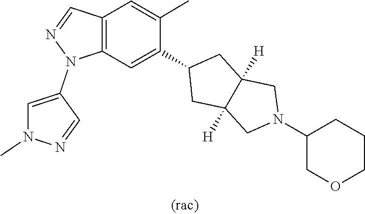 [SMILES: Cc1cc2cnn(-c3cnn(C)c3)c2cc1[C@H]1C[C@@H]2CN(C3CCCOC3)C[C@@H]2C1] | (rac)-5-methyl-1-(1- methyl-1H-pyrazol-4-yl)- 6-((3aR,5s,6aS)-2- (tetrahydro-2H-pyran-3- yl)octahydrocyclopenta[c] pyrrol-5-yl)-1H-indazole | [M + H]⁺ = 406 |
| 12 | 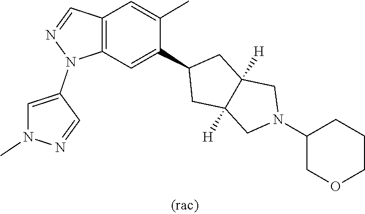 [SMILES: Cc1cc2cnn(-c3cnn(C)c3)c2cc1[C@@H]1C[C@@H]2CN(C3CCCOC3)C[C@@H]2C1] | (rac)-5-methyl-1-(1- methyl-1H-pyrazol-4-yl)- 6-((3aR,5r,6aS)-2- (tetrahydro-2H-pyran-3- yl)octahydrocyclopenta[c] pyrrol-5-yl)-1H-indazole | [M + H]⁺ = 406 |
| 13 | 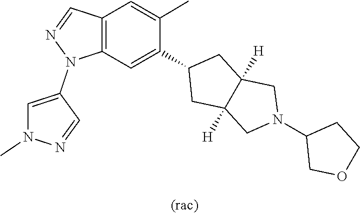 [SMILES: Cc1cc2cnn(-c3cnn(C)c3)c2cc1[C@H]1C[C@@H]2CN(C3CCOC3)C[C@@H]2C1] | (rac)-5-methyl-1-(1- methyl-1H-pyrazol-4-yl)- 6-((3aR,5s,6aS)-2- (tetrahydrofuran-3- yl)octahydrocyclopenta[c] pyrrol-5-yl)-1H-indazole | [M + H]⁺ = 392 |
| 14 | 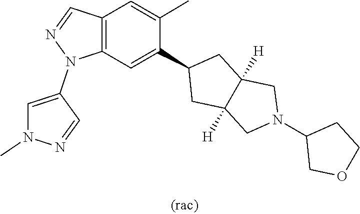 [SMILES: Cc1cc2cnn(-c3cnn(C)c3)c2cc1[C@@H]1C[C@@H]2CN(C3CCOC3)C[C@@H]2C1] | (rac)-5-methyl-1-(1- methyl-1H-pyrazol-4-yl)- 6-((3aR,5r,6aS)-2- (tetrahydrofuran-3- yl)octahydrofuran-3- yl)octahydrocyclopenta[c] pyrrol-5-yl)-1H-indazole | [M + H]⁺ = 392 |
| 15 | 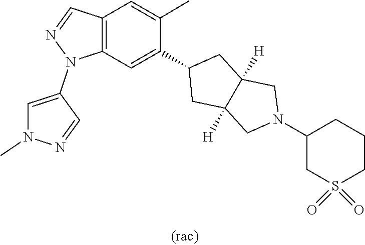 [SMILES: Cc1cc2cnn(-c3cnn(C)c3)c2cc1[C@H]1C[C@@H]2CN(C3CCCS(=O)(=O)C3)C[C@@H]2C1] | (rac)-3-((3aR,5s,6aS)-5- (5-methyl-1-(1-methyl- 1H-pyrazol-4-yl)-1H- indazol-6- yl)hexahydrocyclopenta[c] pyrrol-2(1H)- yl)tetrahydro-2H- thiopyran 1,1-dioxide | ¹H NMR (400 MHz, MeOD) δ 8.07 (s, 1H), 8.02 (d, J = 0.8 Hz, 1H), 7.82 (d, J = 0.5 Hz, 1H), 7.56 (s, 1H), 7.42 (s, 1H), 3.99 (s, 3H), 3.59- 3.48 (m, 1H), 3.36 (m, 1H), 3.14- 2.98 (m, 5H), 2.89-2.77 (m, 3H), 2.48 (s, 3H), 2.27 (ddd, J = 23.1, 9.2, 6.2 Hz, 2H), 2.22-2.10 (m, 2H), 2.00-1.79 (m, 5H), 1.56- 1.44 (m, 1H); [M + H]⁺ = 454 |
| 16 | 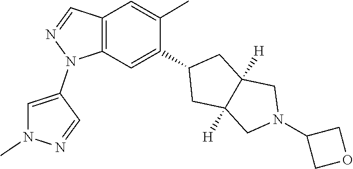 [SMILES: Cc1cc2cnn(-c3cnn(C)c3)c2cc1[C@H]1C[C@@H]2CN(C3COC3)C[C@@H]2C1] | 5-methyl-1-(1-methyl-1H- pyrazol-4-yl)-6- ((3aR,5s,6aS)-2-(oxetan-3- yl)octahydrocyclopenta[c] pyrrol-5-yl)-1H-indazole | [M + H]⁺ = 378 |
| 17 | 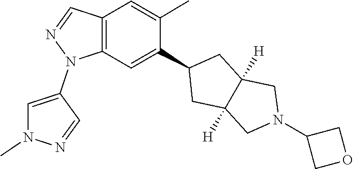 [SMILES: Cc1cc2cnn(-c3cnn(C)c3)c2cc1[C@@H]1C[C@@H]2CN(C3COC3)C[C@@H]2C1] | 5-methyl-1-(1-methyl-1H- pyrazol-4-yl)-6- ((3aR,5r,6aS)-2-(oxetan-3- yl)octahydrocyclopenta[c] pyrrol-5-yl)-1H-indazole | [M + H]⁺ = 378 |
| 18 | 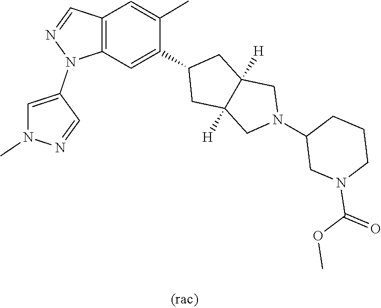 [SMILES: COC(=O)N1CCCC(N2C[C@H]3C[C@H](c4cc5c(cnn5-c5cnn(C)c5)cc4C)C[C@H]3C2)C1] | (rac)-methyl 3- ((3aR,5s,6aS)-5-(5-methyl- 1-(1-methyl-1H-pyrazol-4- yl)-1H-indazol-6- yl)hexahydrocyclopenta[c] pyrrol-2(1H)- yl)piperidine-1- carboxylate | [M + H]⁺ = 463 |
| 19 | 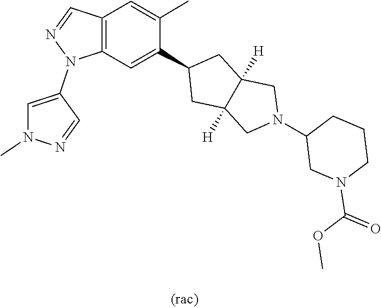 [SMILES: COC(=O)N1CCCC(N2C[C@H]3C[C@@H](c4cc5c(cnn5-c5cnn(C)c5)cc4C)C[C@H]3C2)C1] | (rac)-methyl 3- ((3aR,5r,6aS)-5-(5-methyl- 1-(1-methyl-1H-pyrazol-4- yl)-1H-indazol-6- yl)hexahydrocyclopenta[c] pyrrol-2(1H)- yl)piperidine-1- carboxylate | [M + H]⁺ = 463 |
| 20 | 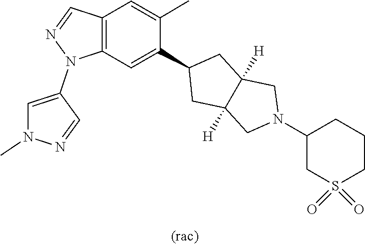 [SMILES: Cc1cc2cnn(-c3cnn(C)c3)c2cc1[C@@H]1C[C@@H]2CN(C3CCCS(=O)(=O)C3)C[C@@H]2C1] | (rac)-3-((3aR,5r,6aS)-5- (5-methyl-1-(1-methyl- 1H-pyrazol-4-yl)-1H- indazol-6- yl)hexahydrocyclopenta[c] pyrrol-2(1H)- yl)tetrahydro-2H- thiopyran 1,1-dioxide | [M + H]⁺ = 454 |

### Commercial Starting Materials

| No. | Structure | Name | CAS # | Supplier |
|---|---|---|---|---|
| 1 | 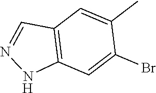 | 6-bromo-5- methyl-1H- indazole | 1000343-69-0 | AmBeed |
| 2 | 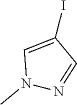 | 4-iodo-1-methyl- 1H-pyrazole | 39806-90-1 | Combi-Blocks, Inc. |
| 3 | 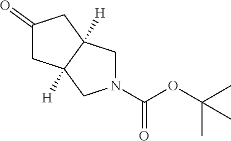 [SMILES: CC(C)(C)OC(=O)N1C[C@H]2CC(=O)C[C@H]2C1] | tert-butyl (3aR,6aS)-5- oxohexahydro- cyclopenta[c] pyrrole-2(1H)- carboxylate | 146231-54-1 | AmBeed BLD Pharmatech Synthonix |
| 4 | 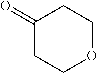 | tetrahydro-4H- pyran-4-one | 29943-42-8 | Combi-Blocks, Inc. Oakwood Products, Inc. |
| 5 | 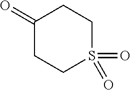 | tetrahydro-4H- thiopyran-4-one 1,1-dioxide | 17396-35-9 | Combi-Blocks, Inc. ChemScene |
| 6 | 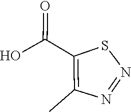 | 4-methyl-1,2,3- thiadiazole-5- carboxylic acid | 18212-21-0 | AmBeed |
| 7 | 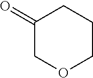 | dihydro-2H- pyran-3(4H)-one | 23462-75-1 | J & W PharmLab AstaTech, Inc |
| 8 | 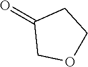 | dihydrofuran- 3(2H)-one | 22929-52-8 | Combi-Blocks, Inc. Ark Pharm, Inc. |
| 9 | 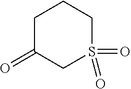 | dihydro-2H- thiopyran-3(4H)- one 1,1-dioxide | 29431-37-6 | J & W PharmLab |
| 10 | 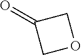 | oxetan-3-one | 6704-31-0 | Combi-Blocks, Inc. Sigma-Aldrich Corporation |
| 11 | 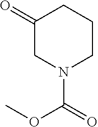 | methyl 3- oxopiperidine-1- carboxylate | 61995-18-4 | AstaTech, Inc |

### Biological Activity

### Biochemical LRRK2 LanthaScreen Assay.

The in-vitro LRRK2 wild type (WT) and G2019S (GS) mutant enzyme activities are assessed for their abilities to phosphorylate a peptide substrate using the LanthaScreen™ (Thermo Fisher) Time-Resolved Fluorescence Energy Transfer (TR-FRET) kinase assay format. The kinase reaction is composed of purified human LRRK2 WT or GS enzymes, a fluorescein labelled peptide substrate (LRRKtide), and ATP. Subsequently, the resulting phosphorylated LRRKtide is recognized by a Terbium-labelled antibody that specifically binds only to the phosphorylated form of LRRKtide. This association leads to an increase in TR-FRET emission ratio of 520 nm/495 nm. In detail, when the phosphorylated LRRKtide is bound to the Terbium (donor) labelled antibody, the FRET signal measured with a 495 nm filter specific to Terbium induces the excitation of the fluorescein (acceptor) of the phosphorylated LRRKtide. Then, this leads to the FRET emission signal measured with a 520 nm filter specific to fluorescein. Therefore, the increase in FRET ratio (520/495) is directly proportional to the activation of LRRK2 kinase activity.

The newly synthesized LRRK2 inhibitors are evaluated for their abilities to block phosphorylation of the LRRKtide substrate by WT and GS mutant enzymes. The level of phosphorylation inhibition is proportional to the level of FRET ratio decrease. The 384 well assay format accommodates a total of 16 inhibitors as duplicates in a single plate, and the activities of inhibitors are measured both at WT and GS mutant in separate plates. Each assay plate also includes 3 reference controls; (1) full kinase activity control wells containing 1% DMSO vehicle, (2) maximum kinase inhibition control wells containing 1 μM MLi-2, well-known LRRK2 inhibitor, and (3) no enzyme control wells serving as assay background level that should match to the maximum enzyme inhibition level by a 1 M MLi-2 control.

Inhibitors (1 mM stock solution in DMSO) are serially diluted 1:4 into 11 point concentration response curves in DMSO using the Bravo Liquid Handler (Agilent). Using an Echo 650 acoustic liquid handler (Beckman Coulter), 100 nl of each solution is transferred to a 384 well Lumitrac 200 assay plate (Greiner, #781075) as 100× to the 10 μL kinase reaction volume per well. Therefore, the final concentration of 11 point dose response curves of inhibitors ranges from 10 μM to 0.01 nM. For both full enzyme control and no enzyme control wells, 100 nL DMSO is added. For maximum enzyme inhibition wells, 100 nL of 0.1 mM MLi-2 is added.

Next, LRRK2 enzymes, substrate, and ATP are diluted as 2× in 1× kinase buffer (50 mM Tris, pH 8.5, 5 mM MgCl₂, 1 mM EGTA, 0.01% BRIJ-35, 2 mM DTT). First, the purified human LRRK2 enzymes are diluted to 6 nM (2×) in a 5 μL volume per well. After adding the WT proteins (Thermo Fisher, cat #PR8604B) and GS mutant proteins (Thermo Fisher, cat #PR8764C) to separate assay plates containing inhibitors/DMSO, the plates are spun down at 100×g for 1 min and placed on a plate shaker with gentle shaking at 50 rpm to incubate enzyme with inhibitors/DMSO at 25° C. for 15 min. The kinase buffer alone (5 μL/well) is added to the no enzyme control wells. Secondly, LRRKtide substrate (Thermo Fisher #PV4901) and ATP (Thermo Fisher #PV3227) are diluted to 2× (0.4 M and 50 μM, respectively) in a 5 μL volume per well. Immediately after the 15 min enzyme incubation, 5 μL of LRRKtide and ATP mixtures are added to entire plates. The plates are spun down at 100×g for 1 min, and covered with a black lid to protect from light. Kinase reaction is allowed at 25° C. for 120 mins with gentle shaking at 50 rpm.

The 2× TR-FRET detection/stop solutions are prepared by adding 0.5 nM terbium-pLRRKtide antibody (Thermo Fisher, #PV4899) and 20 mM EDTA (Thermo Fisher, #15575-020) to the TR-FRET dilution buffer (Thermo Fisher, #PV3574). The kinase reaction is immediately stopped by adding 10 μL of 2× TR-FRET detection solution to entire plates. The plates were spun down at 100×g for 1 min, and covered with a black lid to protect from light. The TR-FRET detection reaction was allowed at 25° C. for 30 mins with gentle shaking at 50 rpm.

Using EnVision Plate reader (Perkin Elmer), TR-FRET signals are measured at two channels, 495 and 520 nm. The fluorescent ratio (520/495) of the full enzyme activity controls with DMSO is converted as 100% enzyme activity, and maximum enzyme inhibition with 1 μM MLi-2 as 0% activity. All ratio data are then normalized to % enzyme activity. Curve fitting and potencies (IC₅₀) of the inhibitors are determined using a four-parameter logistical equation using GraphPad Prism (La Jolla, CA). Inhibitor's selectivity is represented as WT/G2019S ratio by dividing IC₅₀ at WT by IC₅₀ at G2019S.

### Cellular Phospho-LRRK2 S935 HTRF Assay.

The phosphorylation level at Serine 935 of LRRK2 is measured as a readout of cellular LRRK2 kinase activity of both WT and G2019S mutant. The LRRK2 inhibitors are assessed on cellular LRRK2 kinase activity in HEK293 cells stably expressing human LRRK2 WT or G2019S mutant using homogenous time-resolved fluorescence technique (HTRF). This method utilizes two antibodies, europium-Cryptate (donor) antibody and d2-Cryptate (acceptor) antibody, specifically binding to total and phospho S935 of LRRK2, respectively. When two fluorophores are in close proximity, i.e. activation of kinase, time-resolved fluorescence energy transfer (TR-FRET) occurs where a light source excites europium donor, the 615 nm emission by europium then excites d2 acceptor. This leads to the 665 nm emission, resulting in the increase in FRET ratio (665/615). Therefore, FRET ratio (665/615) is directly proportional to LRRK2 kinase activity.

Human LRRK2 WT- or G2019S-HEK293 cells are cultured in DMEM medium containing 10% fetal bovine serum, 2 mM GlutaMax, 100 units/mL antibiotics/antimycotic, and 0.4 mg/mL G418 in 37° C. humidified incubators in the presence of 5% CO₂. All cell culture reagents were purchased from Life Technologies, and phospho-LRRK2 S935 assay reagents were from Perkin Elmer (#6FLRKPEH). Briefly, the day before the assay, the cells (5,000 cells/20 μL/well) are prepared in the plating medium (growth medium without G418). The cells are plated to white solid flat-bottomed, 384 well cell plates (Greiner Bio-One, #78108) that are coated with poly-d-lysine (50 μg/mL), and incubated overnight in 37° C. incubator in the presence of 5% CO₂. Our 384 well assay format accommodates a total of 16 inhibitors as duplicates in a single plate. The activities of inhibitors at WT and GS mutant are measured in separate cell plates.

The next day, inhibitors (2 mM stock) are serially diluted 1:3 into 11 point concentration response curves in DMSO using the Bravo Liquid Handler (Agilent). Using an Echo acoustic liquid handler (Beckman Coulter), 600 nL of each solution is transferred to a 384 well compound plate. For full enzyme control and maximum enzyme inhibition wells, 600 nL of DMSO and 0.2 mM MLi-2 were transferred, respectively. The final 2× compound plate is prepared by adding 60 μL of plating medium to each well. After gently shaking the compound plate on a plate shaker for 1 min, 20 μL of each solution is added directly to the entire cell plate using a 384-tip head of Bravo Liquid Handler. The final concentration of 11 point dose response curves of inhibitors ranges from 10 μM to 0.17 nM. Compounds are incubated for 2 hrs in 37° C. humidified incubators.

During compound incubation, 4× lysis buffer and 100× blocking buffer are diluted to 1× with H₂O. After 2 hr incubation, treatment medium is removed using an ELx405 Microplate washer (Agilent), and 16 μL of lysis/blocking buffer is immediately added to each well in entire plates. The plates are sealed with a plate seal, and placed on a plate shaker for 30 min at room temperature with shaking at 200 rpm.

During the lysis step, 1× antibody working solutions are prepared by diluting 40× Eu-Cryptate antibody and d2-antibody in detection buffer. After the cell plates are spun at 100×g for 1 min, 4 μL of antibody solutions is added to each well in entire plates. The plates are sealed with a plate seal, and placed on a plate shaker for 4 hrs at room temperature with shaking at 200 rpm.

Using EnVision Plate reader (Perkin Elmer), TR-FRET signals are measured at two channels, 615 and 665 nm. The fluorescent ratio (665/615) of the full enzyme activity controls with DMSO is converted as 100% enzyme activity, and maximum enzyme inhibition with 1 μM MLi-2 as 0% activity. All ratio data are then normalized to % enzyme activity. Curve fitting and potencies (IC₅₀) of the inhibitors are determined using a four-parameter logistical equation using GraphPad Prism (La Jolla, CA). Inhibitor's selectivity is represented as WT/G2019S ratio by dividing IC₅₀ at WT by IC₅₀ at G2019S.

TABLE 2
LRRK2 Biochemical Activity Assay

| No. | LRRK2 G2019S IC₅₀ (nM) | LRRK2 WT IC₅₀ (nM) |
|---|---|---|
| 1 | 142.1 | 113 |
| 2 | 5604 | 2293 |
| 3 | 160 | 716 |
| 4 | 30 | 526 |
| 5 | 19.3 | 287 |
| 6 | 2185 | 1861 |
| 7 | 3.5 | 79 |
| 8 | 1254 | 813 |
| 9 | 54.9 | 156 |
| 10 | 672 | 643 |
| 11 | 90 | 247 |
| 12 | 940 | 642 |
| 13 | 16 | 61 |
| 14 | 961 | 885 |
| 15 | 1 | 15 |
| 16 | 10 | 28 |
| 17 | 203 | 281 |
| 18 | 166 | 1528 |
| 19 | 1502 | 1224 |
| 20 | 1662 | 1940 |

TABLE 3
pS935 LRRK2 Cellular Activity Assay

| No. | LRRK2 G2019S IC₅₀ (nM) | LRRK2 WT IC₅₀ (nM) |
|---|---|---|
| 1 | 3626 | 7584 |
| 2 | inactive | inactive |
| 3 | 2982 | >10000 |
| 4 | 2963 | >10000 |
| 5 | 876 | >10000 |
| 6 | >10000 | inactive |
| 7 | 122 | 7332 |
| 8 | >10000 | inactive |
| 9 | 4893 | 8434 |
| 10 | 9136 | inactive |
| 11 | 2313 | 4339 |
| 12 | 8721 | inactive |
| 13 | 482 | 8732 |
| 14 | 5527 | >10000 |
| 15 | 8.1 | 1490 |
| 16 | 108 | 1640 |
| 17 | 2202 | >10000 |
| 18 | 4107 | inactive |
| 19 | inactive | inactive |
| 20 | >10000 | inactive |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A compound of formula (I): 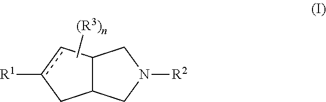
or a pharmaceutically acceptable salt thereof, wherein:
“ [symbol] ” represents a single bond or a double bond;
R¹ is 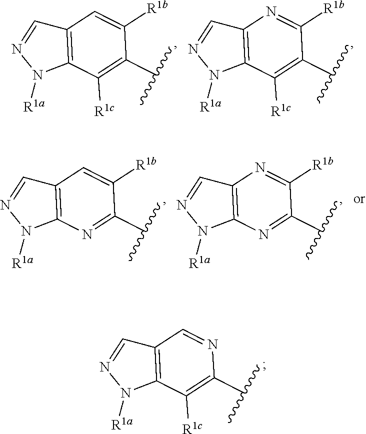
R^{1a} is G¹, -L¹-G¹, —C₂₋₄alkylene-OR^{A}, —C₂₋₄alkylene-NR^{A}R^{B}, C₁₋₆alkyl, C₁₋₆fluoroalkyl, or H;
R^{A} is G¹, -L¹-G¹, C₁₋₆alkyl, C₁₋₆fluoroalkyl, C₃₋₆cycloalkyl, or —C₁₋₃alkylene-C₃₋₆cycloalkyl;
R^{B} is H, C₁₋₆alkyl, C₁₋₆fluoroalkyl, C₃₋₆cycloalkyl, or —C₁₋₃alkylene-C₃₋₆cycloalkyl;
R^{1b} is halogen, C₁₋₄alkyl, C₁₋₂fluoroalkyl, CN, C₃₋₄cycloalkyl, —OC₁₋₃alkyl, —OC₁₋₂fluoroalkyl, or H;
R^{1c} is H, halogen, C₁₋₄alkyl, C₁₋₂fluoroalkyl, CN, phenyl, C₃₋₄cycloalkyl, —OR^{1d}, or —N(R^{1d})₂;
R^{1d}, at each occurrence, is independently H, C₁₋₄alkyl, C₁₋₂fluoroalkyl, C₃₋₄cycloalkyl, or —C₁₋₃alkylene-C₃₋₄cycloalkyl, wherein alternatively two R^{1d}, together with a nitrogen to which the two R^{1d} attach form a 4- to 6-membered heterocyclic ring optionally substituted with 1-4 substituents independently selected from the group consisting of halogen and C₁₋₄alkyl;
L¹ is C₁₋₅alkylene or C₂₋₅alkenylene;
G¹ is a 5- to 6-membered aromatic or partially unsaturated heterocyclic ring containing a first nitrogen and optionally 1-2 additional heteroatoms that are independently nitrogen, oxygen, or sulfur, the heterocyclic ring being attached at an unsaturated carbon in the heterocyclic ring or G¹ is phenyl, G¹ being optionally substituted with a first substituent selected from the group consisting of halogen, C₁₋₄alkyl, cyano, C₁₋₂fluoroalkyl, oxo, —OR¹⁰, —N(R¹⁰)₂, —C₁₋₃alkylene-OR¹⁰, C₃₋₅cycloalkyl, and —C₁₋₃alkylene-C₃₋₅cycloalkyl, and optionally further substituted with 1-2 substituents independently selected from the group consisting of halogen, C₁₋₄alkyl, cyano, and C₁₋₂fluoroalkyl;
R¹⁰, at each occurrence, is independently H, C₁₋₄alkyl, C₁₋₂fluoroalkyl, C₃₋₄cycloalkyl, or —C₁₋₃alkylene-C₃₋₄cycloalkyl, wherein alternatively two R¹⁰, together with a nitrogen to which the two R¹⁰ attach form a 4- to 6-membered heterocyclic ring optionally substituted with 1-4 substituents independently selected from the group consisting of halogen and C₁₋₄alkyl;
R² is G², -L²-G², -L³-C₁₋₆alkyl, —C₂₋₆alkylene-R^{2a}, C₁₋₆alkyl, C₁₋₆fluoroalkyl, or H;
L² is C₁₋₃alkylene or L³;
L³ is C(O), SO₂, S(O)(NH), C(O)NH, or C(O)O;
G² is a 4- to 12-membered heterocyclyl, a C₃₋₁₂carbocyclyl, a 5- to 12-membered heteroaryl, or a 6- to 12-membered aryl, wherein G² is optionally substituted with a first substituent selected from the group consisting of halogen, cyano, C₁₋₄alkyl, C₁₋₂fluoroalkyl, G^{2a}, oxo, —OR¹³, —N(R¹³)₂, —C₁₋₃alkylene-OR¹³, —C₁₋₃alkylene-N(R¹³)₂, —C(O)N(R¹³)₂, —C(O)OR¹³, —SO₂R¹³ and S(O)(NH)R¹³, optionally further substituted with oxo, and optionally further substituted with 1-3 substituents independently selected from the group consisting of halogen, cyano, C₁₋₄alkyl, and C₁₋₂fluoroalkyl;
G^{2a} is a C₃₋₄cycloalkyl;
R¹³, at each occurrence, is independently H, C₁₋₄alkyl, C₁₋₂fluoroalkyl, C₃₋₄cycloalkyl, or —C₁₋₃alkylene-C₃₋₄cycloalkyl, wherein alternatively two R¹³, together with a nitrogen to which the two R¹³ attach form a 4- to 6-membered heterocyclic ring optionally substituted with 1-4 substituents independently selected from the group consisting of halogen and C₁₋₄alkyl;
R^{2a} is —OR¹⁴, —N(R¹⁴)₂, —SO₂R¹⁴, S(O)(NH)R¹⁴, —NR¹⁴C(O)N(R¹⁴)₂, or —NR¹⁴C(O)OR¹⁴;
R¹⁴, at each occurrence, is independently H, C₁₋₄alkyl, C₁₋₂fluoroalkyl, C₃₋₄cycloalkyl, or —C₁₋₃alkylene-C₃₋₄cycloalkyl, wherein alternatively two R¹⁴, together with a nitrogen to which the two R¹⁴ attach form a 4- to 6-membered heterocyclic ring optionally substituted with 1-4 substituents independently selected from the group consisting of halogen and C₁₋₄alkyl;
R³, at each occurrence, is independently fluoro or C₁₋₄alkyl; and
n is 0, 1, or 2.

2. The compound of claim 1, or a pharmaceutically acceptable salt thereof, wherein R^{1a} is G¹; and G¹ is the optionally substituted 5- to 6-membered aromatic heterocyclic ring.

3. The compound of claim 1, or a pharmaceutically acceptable salt thereof, wherein the ring system of the optionally substituted 5- to 6-membered aromatic heterocyclic ring at G¹ contains 1-2 nitrogens.

4. The compound of claim 1, or a pharmaceutically acceptable salt thereof, wherein G¹ is optionally substituted with a first substituent selected from the group consisting of halogen, C₁₋₄alkyl, cyano, C₁₋₂fluoroalkyl, —OR¹⁰, C₃₋₅cycloalkyl, and —C₁₋₃alkylene-C₃₋₅cycloalkyl, and further optionally substituted with 1-2 substituents independently selected from the group consisting of halogen and C₁₋₄alkyl.

5. The compound of claim 1, or a pharmaceutically acceptable salt thereof, wherein R^{1a} is G¹; and G¹ is the optionally substituted 5- to 6-membered partially unsaturated heterocyclic ring.

6. The compound of claim 1, or a pharmaceutically acceptable salt thereof, wherein the ring system of the optionally substituted 5- to 6-membered partially unsaturated heterocyclic ring is 1,2-dihydropyridin-4-yl.

7. The compound of claim 1, or a pharmaceutically acceptable salt thereof, wherein G¹ is

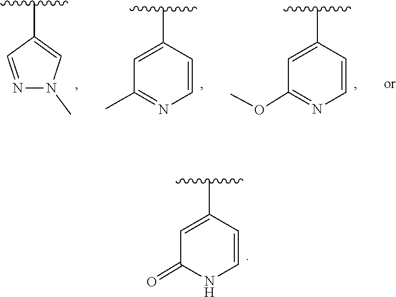

8. The compound of claim 1, or a pharmaceutically acceptable salt thereof, wherein R^{1b} is halogen or C₁₋₄alkyl.

9. The compound of claim 1, or a pharmaceutically acceptable salt thereof, wherein R^{1c} is H.

10. The compound of claim 1, or a pharmaceutically acceptable salt thereof, wherein R² is G² or -L²-G².

11. (canceled)

12. The compound of claim 1, or a pharmaceutically acceptable salt thereof, wherein L² is C₁₋₃alkylene, or wherein L² is L³ and L³ is C(O).

13. (canceled)

14. The compound of claim 1, or a pharmaceutically acceptable salt thereof, wherein G² is the optionally substituted 4- to 12-membered heterocyclyl or the optionally substituted C₃₋₁₂carbocyclyl.

15. The compound of claim 14, or a pharmaceutically acceptable salt thereof, wherein the ring system of the optionally substituted 4- to 12-membered heterocyclyl at G² is a 4- to 8-membered heterocyclyl or an 8- to 10-membered fused bicyclic heterocyclyl, the heterocyclyls containing 1-2 heteroatoms independently selected from the group consisting of oxygen, nitrogen, and sulfur.

16. (canceled)

17. The compound of claim 14, or a pharmaceutically acceptable salt thereof, wherein the ring system of the optionally substituted C₃₋₁₂carbocyclyl at G² is a C₃₋₆cycloalkyl.

18. The compound of claim 1, or a pharmaceutically acceptable salt thereof, wherein G² is the optionally substituted 5- to 12-membered heteroaryl or the optionally substituted 6- to 12-membered aryl.

19. The compound of claim 18, or a pharmaceutically acceptable salt thereof, wherein the ring system of the optionally substituted 5- to 12-membered heteroaryl at G² is a 5- to 6-membered heteroaryl containing 1-3 heteroatoms independently selected from the group consisting of oxygen, nitrogen, and sulfur.

20. (canceled)

21. The compound of claim 1, or a pharmaceutically acceptable salt thereof, wherein G² is

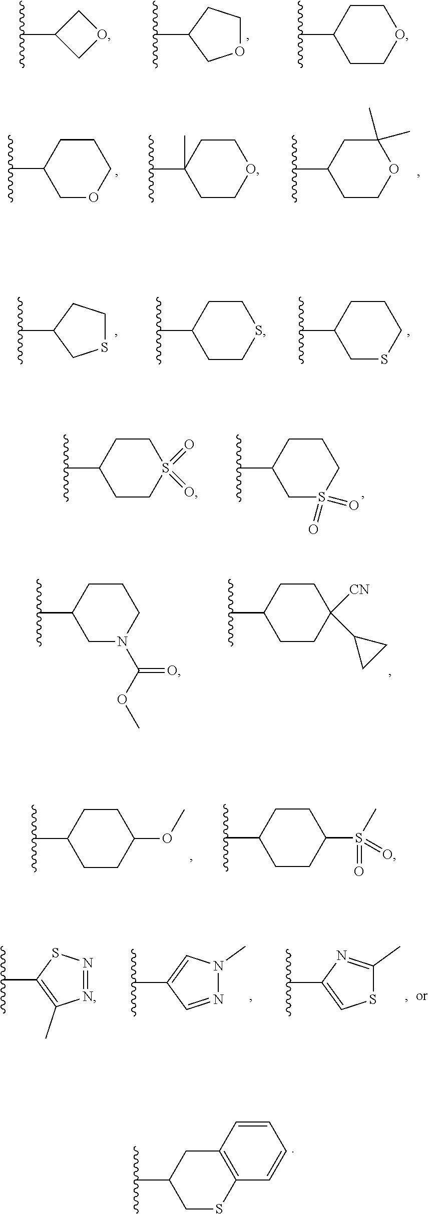

22. The compound of claim 1, or a pharmaceutically acceptable salt thereof, wherein n is 0.

23. The compound of claim 1, or a pharmaceutically acceptable salt thereof, wherein R¹ is

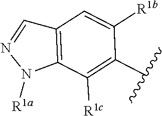

24. The compound of claim 1, or a pharmaceutically acceptable salt thereof, wherein the compound has formula (II-C) or formula (III-C):

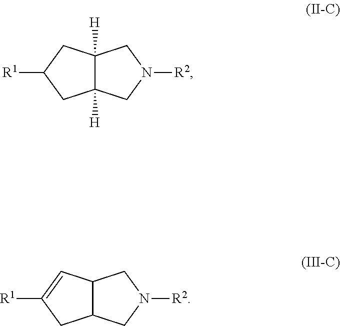

25. (canceled)

26. The compound of claim 1 selected from the group consisting of:
6-((3aR,6aS)-1,2,3,3a,4,6a-hexahydrocyclopenta[c]pyrrol-5-yl)-5-methyl-1-(1-methyl-1H-pyrazol-4-yl)-1H-indazole;
tert-butyl (3aR,6aS)-5-(5-methyl-1-(1-methyl-1H-pyrazol-4-yl)-1H-indazol-6-yl)-3,3a,4,6a-tetrahydrocyclopenta[c]pyrrole-2(1H)-carboxylate;
5-methyl-1-(1-methyl-1H-pyrazol-4-yl)-6-((3aR,6aS)-2-(tetrahydro-2H-pyran-4-yl)-1,2,3,3a,4,6a-hexahydrocyclopenta[c]pyrrol-5-yl)-1H-indazole;
4-((3aR,6aS)-5-(5-methyl-1-(1-methyl-1H-pyrazol-4-yl)-1H-indazol-6-yl)-3,3a,4,6a-tetrahydrocyclopenta[c]pyrrol-2(1H)-yl)tetrahydro-2H-thiopyran 1,1-dioxide;
5-methyl-1-(1-methyl-1H-pyrazol-4-yl)-6-((3aR,5s,6aS)-2-(tetrahydro-2H-pyran-4-yl)octahydrocyclopenta[c]pyrrol-5-yl)-1H-indazole;
5-methyl-1-(1-methyl-1H-pyrazol-4-yl)-6-((3aR,5r,6aS)-2-(tetrahydro-2H-pyran-4-yl)octahydrocyclopenta[c]pyrrol-5-yl)-1H-indazole;
4-((3aR,5s,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide;
4-((3aR,5r,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide;
(4-methyl-1,2,3-thiadiazol-5-yl)((3aR,5s,6aS)-5-(5-methyl-1-(1-methyl-1H-pyrazol-4-yl)-1H-indazol-6-yl)hexahydrocyclopenta[c]pyrrol-2(1H)-yl)methanone;
(4-methyl-1,2,3-thiadiazol-5-yl)((3aR,5r,6aS)-5-(5-methyl-1-(1-methyl-1H-pyrazol-4-yl)-1H-indazol-6-yl)hexahydrocyclopenta[c]pyrrol-2(1H)-yl)methanone;
5-methyl-1-(1-methyl-1H-pyrazol-4-yl)-6-((3aR,5s,6aS)-2-(tetrahydro-2H-pyran-3-yl)octahydrocyclopenta[c]pyrrol-5-yl)-1H-indazole;
5-methyl-1-(1-methyl-1H-pyrazol-4-yl)-6-((3aR,5r,6aS)-2-(tetrahydro-2H-pyran-3-yl)octahydrocyclopenta[c]pyrrol-5-yl)-1H-indazole;
5-methyl-1-(1-methyl-1H-pyrazol-4-yl)-6-((3aR,5s,6aS)-2-(tetrahydrofuran-3-yl)octahydrocyclopenta[c]pyrrol-5-yl)-1H-indazole;
5-methyl-1-(1-methyl-1H-pyrazol-4-yl)-6-((3aR,5r,6aS)-2-(tetrahydrofuran-3-yl)octahydrocyclopenta[c]pyrrol-5-yl)-1H-indazole;
3-((3aR,5s,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide;
5-methyl-1-(1-methyl-1H-pyrazol-4-yl)-6-((3aR,5s,6aS)-2-(oxetan-3-yl)octahydrocyclopenta[c]pyrrol-5-yl)-1H-indazole;
5-methyl-1-(1-methyl-1H-pyrazol-4-yl)-6-((3aR,5r,6aS)-2-(oxetan-3-yl)octahydrocyclopenta[c]pyrrol-5-yl)-1H-indazole;
methyl 3-((3aR,5s,6aS)-5-(5-methyl-1-(1-methyl-1H-pyrazol-4-yl)-1H-indazol-6-yl)hexahydrocyclopenta[c]pyrrol-2(1H)-yl)piperidine-1-carboxylate;
methyl 3-((3aR,5r,6aS)-5-(5-methyl-1-(1-methyl-1H-pyrazol-4-yl)-1H-indazol-6-yl)hexahydrocyclopenta[c]pyrrol-2(1H)-yl)piperidine-1-carboxylate;
3-((3aR,5r,6aS)-5-(5-methyl-1-(1-methyl-1H-pyrazol-4-yl)-1H-indazol-6-yl)hexahydrocyclopenta[c]pyrrol-2(1H)-yl)tetrahydro-2H-thiopyran 1,1-dioxide;
or a pharmaceutically acceptable salt thereof.

27. A pharmaceutical composition comprising the compound of claim 1, or a pharmaceutically acceptable salt thereof, and a pharmaceutically acceptable carrier.

28. A method of treating a disease or disorder comprising administering to a subject in need thereof a therapeutically effective amount of the compound of claim 1, or a pharmaceutically acceptable salt thereof, wherein the disease or disorder is a CNS disease or disorder, an immunological disease or disorder, a cancer, or leprosy.

29. The method of claim 28, wherein the disease or disorder is:
a CNS disease or disorder and the CNS disease or disorder is selected from the group consisting of familial/genetic and/or sporadic Parkinson's disease, a tauopathy, Alzheimer's disease, neuroinflammation, HIV-induced dementia, ALS, ischemic stroke, traumatic brain injury and spinal cord injury; or
an immunological disease or disorder and the immunological disease or disorder is selected from the group consisting of lymphomas, leukemias, multiple sclerosis, rheumatoid arthritis, systemic lupus erythematosus, autoimmune hemolytic anemia, pure red cell aplasia, idiopathic thrombocytopenic pupura (ITP), Evans Syndrome, vasculitis, bullous skin disorder, type I diabetes mellitus, Sjogren's syndrome, Delvic's disease, inflammatory myopathies, ankylosing spondylitis, and Crohn's disease; or
a cancer and the cancer is selected from the group consisting of renal cancer, breast cancer, lung cancer, prostate cancer, acute myelogenous leukemia (AML), papillary renal carcinoma, thyroid carcinoma, and low-grade glioma

30-31. (canceled)

32. The method of claim 28, wherein the disease or disorder is leprosy.

33. A method of inhibiting LRRK2 in a subject comprising administering to the subject an amount effective to inhibit LRRK2 of the compound of claim 1, or a pharmaceutically acceptable salt thereof.

34. The method of claim 33, wherein LRRK2 is the G2019S mutant of LRRK2.

35-38. (canceled)
